(19) United States
(12) Patent Application Publication
(10) Pub. No.: US 2008/0114364 A1
(43) Pub. Date: May 15, 2008

(54) Tissue cavitation device and method

(75) Inventors: Mark Goldin, Orlando, FL (US); Brian Schumacher, Orlando, FL (US)

Correspondence Address: FROST BROWN TODD, LLC, 2200 PNC CENTER, 201 E. FIFTH STREET, CINCINNATI, OH, 45202, US

(73) Assignee: AOI Medical, Inc., Orlando, FL (US)

(21) Appl. No.: 11/600,313

(22) Filed: Nov. 15, 2006

(51) Int. Cl. A61B 17/32

(52) U.S. Cl. 606/79; 606/170

(57) ABSTRACT

Provided is a medical device for forming or modifying cavities in tissue. Versions include a blade extendable laterally from a first shape to a second shape to cut tissue in a controlled manner. The first shape may be configured for insertion in accordance with minimally invasive procedures and the second shape may be configured for cutting or forming cavities.

### BACKGROUND

Versions of the present invention relate to orthopedic medical procedures and, more particularly, to forming or modifying cavities within bone tissue for use during orthopedic procedures.

Increasingly, surgeons are using minimally invasive surgical techniques for the treatment of a wide variety of medical conditions. Such techniques typically involve the insertion of a surgical device through a natural body orifice or through a relatively small incision using a tube or cannula. In contrast, conventional surgical techniques typically involve a significantly larger incision and are, therefore, sometimes referred to as open surgery. Thus, as compared with conventional techniques, minimally invasive surgical techniques offer the advantages of minimizing trauma to healthy tissue, minimizing blood loss, reducing the risk of complications such as infection, and reducing recovery time. Further, certain minimally invasive surgical techniques may be performed under local anesthesia or even, in some cases, without anesthesia, and therefore enable surgeons to treat patients who would not tolerate the general anesthesia required by conventional techniques.

Surgical procedures often require the formation of a cavity within either soft or hard tissue, including bone. Tissue cavities are formed for a wide variety of reasons, such as for the removal of diseased tissue, for harvesting tissue in connection with a biopsy or autogenous transplant, and for implant fixation. To achieve the benefits associated with minimally invasive techniques, tissue cavities are generally formed by creating only a relatively small access opening in the target tissue. An instrument or device may then be inserted through the opening and used to form a hollow cavity that is significantly larger than the access opening.

One important surgical application utilizing the formation of a cavity within tissue is the surgical treatment and prevention of skeletal fractures associated with osteoporosis, which is a metabolic disease characterized by a decrease in bone mass and strength. The disease frequently leads to skeletal fractures under light to moderate trauma and, in its advanced state, can lead to fractures under normal physiologic loading conditions. It is estimated that osteoporosis affects approximately 15-20 million people in the United States and that approximately 1.3 million new fractures each year are associated with osteoporosis, with the most common fracture sites being the hip, wrist and vertebrae.

An emerging prophylactic treatment for osteoporosis, trauma, or the like involves replacing weakened bone with a stronger synthetic bone substitute using minimally invasive surgical procedures. The weakened bone is first surgically removed from the affected site, thereby forming a cavity. The cavity is then filled with an injectable synthetic bone substitute and allowed to harden. The synthetic bone substitute provides structural reinforcement and thus lessens the risk of fracture of the affected bone. Without the availability of minimally invasive surgical procedures, however, the prophylactic fixation of osteoporosis-weakened bone in this manner would not be practical because of the increased morbidity, blood loss and risk of complications associated with conventional procedures. Moreover, minimally invasive techniques tend to preserve more of the remaining structural integrity of the bone because they minimize surgical trauma to healthy tissue.

Other less common conditions in which structural reinforcement of bone may be appropriate include bone cancer and avascular necrosis. Surgical treatment for each of these conditions can involve removal of the diseased tissue by creating a tissue cavity and filling the cavity with a stronger synthetic bone substitute to provide structural reinforcement to the affected bone.

Existing devices for forming a cavity within soft or hard tissue are generally relatively complex assemblies. U.S. Pat. No. 5,445,639 to Kuslich et al. (“Kuslich”) discloses an intervertebral reamer for use in fusing contiguous vertebra. The Kuslich device comprises a cylindrical shaft containing a mechanical mechanism that causes cutting blades to extend axially from the shaft to cut a tissue cavity as the shaft is rotated. The shaft of the Kuslich device has a relatively large diameter in order to house the blade extension mechanism, and therefore it may be necessary to create a relatively large access opening to insert the device into the body. Complex devices may be associated with a relatively high cost. An axially projecting cutting instrument may limit the cutting options available to a user during a procedure.

U.S. Pat. No. 5,928,239 to Mirza (“Mirza”) discloses a percutaneous surgical cavitation device and method useful for forming a tissue cavity in minimally invasive surgery. The Mirza device comprises an elongated shaft and a separate cutting tip that is connected to one end of the shaft by a freely-rotating hinge, as shown in FIG. 1. The cutting tip of the Mirza device rotates outward about the hinge, thereby permitting the device to cut a tissue cavity that is larger than the diameter of the shaft. However, the Mirza device may rely on rotation of the shaft at speeds ranging from 40,000 to 80,000 rpm which cause the cutting tip to rotate outward about the hinge. Such high rotational speeds generally are produced with a high-speed surgical drill and may not be possible with manual actuation. Thus, such devices may not permit the surgeon to exercise the precise control that can be attained through manual rotation while still effectively cutting tissue. There may be a concern for structural failure or loosening of the relatively small hinge assembly at such a high rotational speed when operated in bone. The rotation of very high speed surgical drills, such as from the 40,000-80,000 rpm range, may also generate excessive heat that could damage healthy tissue surrounding the cavity.

U.S. Pat. No. 6,066,154 to Reiley et al. (“Reiley”) discloses an inflatable, balloon-like device for forming a cavity within tissue. The Reiley device is inserted into the tissue and then inflated to form the cavity by compressing surrounding tissue, rather than by cutting away tissue. The Reiley device, however, is not intended to cut tissue, and at least a small cavity must generally be cut or otherwise formed in the tissue in order to initially insert the Reiley device. The inflatable device may also limit the control the user or clinician may have over the shape of the cavity and the compression of bone tissue of varying densities may be difficult.

Thus, a need continues to exist for a tissue cavitation device and method that can form tissue cavities in a minimally invasive manner. A need also exists for a cavitation device that has relatively simple construction and is inexpensive to manufacture, that can be operated either manually or by a powered surgical drill, and that provides the user with increased control over the size and shape of the cavity formed.

### BRIEF DESCRIPTION OF THE FIGURES

It is believed the present invention will be better understood from the following description taken in conjunction with the accompanying drawings. The drawings and detailed description that follow are intended to be merely illustrative and are not intended to limit the scope of the invention.

FIG. 1 is a sectional view of the proximal end of the human femur and shows the prior art cavitation device disclosed in U.S. Pat. No. 5,928,239 to Mirza.

FIG. 2A is a perspective view showing one version of a cavitation device attached to a surgical drill.

FIG. 2B is a more detailed view of the distal end of the cavitation device depicted in FIG. 2A showing a flexible cutting element.

FIG. 3A is a perspective view of one version of a flexible cutting element shown in an open position.

FIG. 3B is a longitudinal cross-sectional view of an insertion tube having an aperture shown with the flexible cutting element of FIG. 3A sheathed therein.

FIG. 3C is a longitudinal cross-sectional view of the insertion tube of FIG. 3B shown with the flexible cutting element of FIG. 3B opened through the aperture.

FIG. 4A is a perspective view of one version of a flexible cutting element shown in a resting or first shape.

FIG. 4B is a longitudinal cross-sectional view of an insertion tube having a lateral aperture and a distal aperture shown with the flexible cutting element of FIG. 4A sheathed therein.

FIG. 4C is a longitudinal cross-sectional view of the insertion tube of FIG. 4B shown with the flexible cutting element of FIG. 4B opened through the lateral aperture.

FIG. 4D is a longitudinal cross-sectional view of the insertion tube of FIG. 4B shown with the flexible cutting element of FIG. 4B opened through the distal aperture.

FIG. 5A is a longitudinal cross-sectional view of an insertion tube shown with a flexible cutting element having serrations, cutting flutes, an irrigation passage, and a combined distal and lateral aperture.

FIG. 5B is an alternate longitudinal cross-sectional view of the insertion tube and the flexible cutting element of FIG. 5A more clearly illustrating the combined distal and lateral aperture.

FIG. 6 is a longitudinal cross-sectional view of an insertion tube having an aperture shown with one version of a flexible cutting element opened therethrough.

FIG. 7A is a longitudinal cross-sectional view of an insertion tube, having a first aperture and a second aperture, shown with a flexible cutting element, having a first cutting element and a second cutting element, sheathed therein.

FIG. 7B is a longitudinal cross-sectional view of the insertion tube and flexible cutting element of FIG. 7A shown with the first cutting element and the second cutting element opened through the first aperture and the second aperture, respectively.

FIG. 8A is a longitudinal cross-sectional view of an insertion tube having an aperture with one version of a flexible cutting element opened therethough, where the range of motion of the flexible cutting element is shown.

FIG. 8B is a front cross-sectional view, taken along line 8B-8B, of the insertion tube and flexible element of FIG. 8A, where the shaft of the flexible element is shown in cross-section to display elements configured therein.

FIG. 9A is a partial perspective view of an insertion tube having four apertures and a cavitation device having four flexible cutting elements, where the cavitation device is shown retained within the insertion tube.

FIG. 9B is a partial perspective view of the insertion tube and the cavitation device of FIG. 9A, where the four flexible cutting elements are shown opened through the four apertures.

FIG. 10A is a cross-sectional view of an insertion tube, having a first aperture and a second aperture, and a cavitation device, having a first flexible cutting element and a second cutting element, shown coupled with a T-handle for operation.

FIG. 10B is a more detailed view of the cavitation device of FIG. 10A shown in the opened position as an actuator connected thereto is drawn proximally.

FIG. 11A is a side view of one version of an insertion tube having an aperture inserted into a region of bone tissue, where a flexible element is maintained within the insertion tube.

FIG. 11B is a side view of the cavitation device of FIG. 11A, where the flexible cutting element is shown opened laterally through the aperture of the insertion tube, where the flexible cutting element is shown rotating such that a cavity is created.

FIG. 11C is a side view of the cavity shown in FIG. 11B, where a lumen is shown filling the cavity with a structural compound after the cavity has been cleared.

FIG. 12A is a partial perspective view of an insertion tube having a flexible cutting element maintained therein, where the flexible element is shown in a resting or first shape.

FIG. 12B is a partial perspective view of the insertion tube and flexible cutting element of FIG. 12A, where the flexible cutting element is shown in a second position or shape.

FIG. 12C is a partial perspective view of the insertion tube and flexible cutting element of FIG. 12A, where the flexible cutting element is shown in a third position or shape.

FIG. 13 is a partial perspective view of an insertion tube having an aperture, where a portion of a flexible cutting element is shown extended distally beyond the end of the insertion tube.

FIG. 14A is a partial perspective view of one version of an insertion tube having an aperture with one version of a wound or coiled flexible cutting member maintained therein.

FIG. 14B is a partial perspective view of one version of the insertion tube and flexible cutting member of FIG. 14A, where the flexible cutting member is shown unwound and opened through the aperture of the insertion tube.

FIG. 14C is a top view of the insertion tube and flexible member of FIG. 14B shown unwound, where the flexible cutting element is offset from the longitudinal axis and is shown having a convex cutting surface edge and a concave cutting edge.

FIG. 14D is a side view of the insertion tube and flexible member of FIG. 14B.

FIG. 15A is a partial perspective view of one version of a flexible cutting element and a support member coupled with a tip shown in a resting or first shape.

FIG. 15B is a partial perspective view of the flexible cutting element and the support member of FIG. 15A shown in a second shape opened through a lateral aperture of an insertion tube.

FIG. 15C is a partial perspective view of the flexible cutting element and the support member of FIG. 15A shown in a second shape opened through a distal aperture of an insertion tube.

FIG. 16 is a partial top view of one version of a flexible cutting element.

FIG. 17 is a partial top view of an alternate version of a flexible cutting element.

FIG. 18 is a partial top view of an alternate version of a flexible cutting element.

FIG. 19 is a partial top view of an alternate version of a flexible cutting element.

FIG. 20 is a partial top view of an alternate version of a flexible cutting element.

FIG. 21 is a cross-sectional view taken along line D-D of FIG. 16 of one version of a flexible cutting element.

FIG. 22 is a cross-sectional view of an alternate version of a flexible cutting element.

FIG. 23a is a partial perspective view of one version of a flexible cutting element.

FIG. 23b is a cross-sectional view, taken along line 23a-23a, of the flexible cutting element shown in FIG. 23a.

FIG. 24 is a cross-sectional view of an alternate version of a flexible cutting element.

FIG. 25 is a cross-sectional view of an alternate version of a flexible cutting element.

FIG. 26 is a cross-sectional view of an alternate version of a flexible cutting element.

FIG. 27 is a cross-sectional view of an alternate version of a flexible cutting element.

FIG. 28 is a cross-sectional view of an alternate version of a flexible cutting element.

FIG. 29 is a cross-sectional view of an alternate version of a flexible cutting element.

FIG. 30 is a cross-sectional view of an alternate version of a flexible cutting element.

FIG. 31 is a cross-sectional view of an alternate version of a flexible cutting element.

FIG. 32 is a cross-sectional view of an alternate version of a flexible cutting element.

FIG. 33 is a cross-sectional view of an alternate version of a flexible cutting element.

FIG. 34 is a cross-sectional view of an alternate version of a flexible cutting element.

FIG. 35 is a cross-sectional view of an alternate version of a flexible cutting element.

FIG. 36 is a cross-sectional view of an alternate version of a flexible cutting element.

FIG. 37 is a cross-sectional view of an alternate version of a flexible cutting element.

FIG. 38 is a cross-sectional view of an alternate version of a flexible cutting element.

FIG. 39 is a cross-sectional view of an alternate version of a flexible cutting element.

FIG. 40 is a cross-sectional view of an alternate version of a flexible cutting element.

FIG. 41 is a perspective partial view of one version of a flexible cutting element with a top surface having a plurality of cutting elements.

FIG. 42 is a perspective partial view of one version of a flexible cutting element with a top surface having a plurality of cutting elements.

FIG. 43 is a perspective partial view of one version of a flexible cutting element with a textured top surface.

FIG. 44 is a cross-sectional view of one version of a tissue cavity taken along reference axis A-A shown in FIG. 11B.

FIG. 45 is a cross-sectional view of an alternate version of a tissue cavity shown with reference to axis A-A shown in FIG. 11B.

FIG. 46 is a cross-sectional view of an alternate version of a tissue cavity shown with reference to axis A-A shown in FIG. 11B.

FIG. 47 is a cross-sectional view of an alternate version of a tissue cavity shown with reference to axis A-A shown in FIG. 11B.

FIG. 48 is a cross-sectional view of an alternate version of a tissue cavity shown with reference to axis A-A shown in FIG. 11B.

FIG. 49 is a cross-sectional view of an alternate version of a tissue cavity shown with reference to axis A-A shown in FIG. 11B.

FIG. 50 is a cross-sectional view of an alternate version of a tissue cavity shown with reference to axis A-A shown in FIG. 11B.

FIG. 51 is a cross-sectional view of an alternate version of a tissue cavity shown with reference to axis A-A shown in FIG. 11B.

FIG. 52 is a cross-sectional view of an alternate version of a tissue cavity shown with reference to axis A-A shown in FIG. 11B.

FIG. 53 is a cross-sectional view of an alternate version of a tissue cavity shown with reference to axis A-A shown in FIG. 11B.

FIG. 54 is a cross-sectional view of an alternate version of a tissue cavity shown with reference to axis A-A shown in FIG. 11B.

FIG. 55 is a cross-sectional view of an alternate version of a tissue cavity shown with reference to axis A-A shown in FIG. 11B.

FIG. 56 is a cross-sectional view of an alternate version of a tissue cavity shown with reference to axis A-A shown in FIG. 11B.

FIG. 57 is a perspective view of one version of a combined tissue cavity having cavity sections formed about axis A-A, axis B-B, and axis C-C.

FIG. 58 is a cross-sectional view of an alternate version of a combined tissue cavity taken along line E-E of FIG. 57.

FIG. 59 is a cross-sectional view of an alternate version of a combined tissue cavity taken along line E-E of FIG. 57.

FIG. 60 is a cross-sectional view of an alternate version of a combined tissue cavity taken along line E-E of FIG. 57.

FIG. 61 is a cross-sectional view of an alternate version of a combined tissue cavity taken along line E-E of FIG. 57.

FIG. 62 is a cross-sectional view of an alternate version of a combined tissue cavity taken along line E-E of FIG. 57.

FIG. 63 is a longitudinal cross-sectional view of one version of a tissue cavity shown with reference to axis A-A of FIG. 57.

FIG. 64 is a longitudinal cross-sectional view of an alternate version of a tissue cavity shown with reference to axis A-A of FIG. 57.

FIG. 65 is a longitudinal cross-sectional view of an alternate version of a tissue cavity shown with reference to axis A-A of FIG. 57.

FIG. 66 is a longitudinal cross-sectional view of an alternate version of a tissue cavity shown with reference to axis A-A of FIG. 57.

FIG. 67 is a longitudinal cross-sectional view of an alternate version of a tissue cavity shown with reference to axis A-A of FIG. 57.

FIG. 68 is a longitudinal cross-sectional view of an alternate version of a tissue cavity shown with reference to axis A-A of FIG. 57.

FIG. 69 is a longitudinal cross-sectional view of an alternate version of a tissue cavity shown with reference to axis A-A of FIG. 57.

FIG. 70 is a longitudinal cross-sectional view of a portion of an insertion tube having a flexible cutting element associated therewith.

FIG. 71 is a longitudinal cross-sectional view of a portion of an alternate version of an insertion tube having a flexible cutting element associated therewith.

FIG. 72 is a longitudinal cross-sectional view of a portion of an alternate version of an insertion tube having a flexible cutting element associated therewith.

FIG. 73 is a longitudinal cross-sectional view of a portion of an alternate version of an insertion tube having a flexible cutting element associated therewith.

FIG. 74 is a longitudinal cross-sectional view of a portion of an alternate version of an insertion tube having a flexible cutting element associated therewith.

FIG. 75 is a longitudinal cross-sectional view of a portion of an alternate version of an insertion tube having a flexible cutting element associated therewith.

FIG. 76 is a partial perspective view of a portion of an alternate version of an insertion tube having a flexible cutting element associated therewith.

FIG. 77 is a cross-sectional view of one version of an insertion tube.

FIG. 78 is a cross-sectional view of one version of an insertion tube.

FIG. 79 is a perspective view of one version of a cavitation device.

FIG. 80 is a perspective view of an alternate version of a cavitation device.

FIG. 81 is a perspective view of an alternate version of a cavitation device.

FIG. 82 is a perspective view of an alternate version of a cavitation device.

FIG. 83 is a perspective view of an alternate version of a cavitation device.

FIG. 84 is a perspective view of an alternate version of a cavitation device.

FIG. 85 is a perspective view of an alternate version of a cavitation device.

FIG. 86 is a perspective view of an alternate version of a cavitation device.

FIG. 87 is a perspective view of one version of an articulating cavitation device having an end effector.

FIG. 88 is a more detailed view of the end effector of the articulating cavitation device shown in FIG. 87.

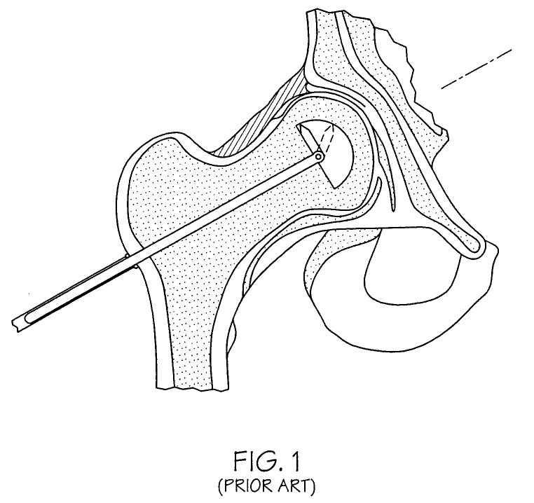

### DETAILED DESCRIPTION OF THE INVENTION

The present invention comprises a tissue cavitation device and method that utilize shape-changing behavior to form or modify cavities in either hard or soft tissue. The shape-changing behavior enables the device to be inserted into tissue through a relatively small access opening, yet also enables the device to form a tissue cavity having a diameter larger than the diameter of the access opening. Thus, the invention may be particularly useful in minimally invasive surgery, and may be used for at least the following specific applications, among others: (1) treatment or prevention of bone fracture, (2) joint fusion, (3) implant fixation, (4) tissue harvesting (especially bone), (5) removal of diseased tissue (hard or soft tissue), (6) general tissue removal (hard or soft tissue) (7) vertebroplasty, and (8) kyphoplasty.

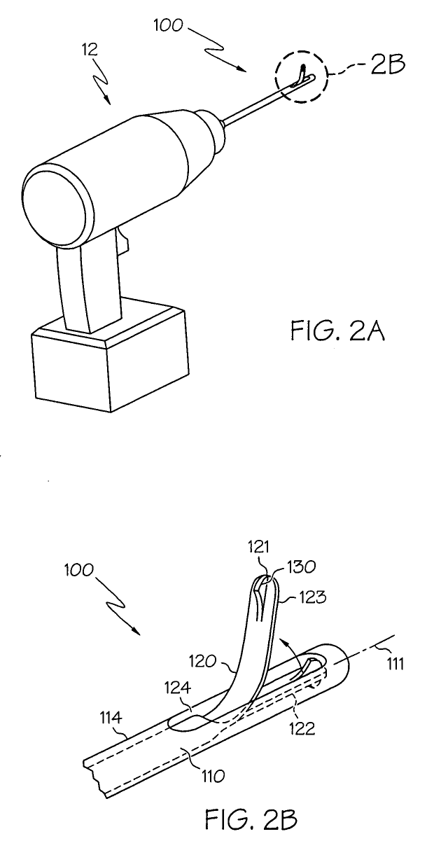

Referring to FIGS. 2-20, versions of the cavitation device of the present invention may include a translatable, rotatable, or movable shaft having a flexible cutting element associated therewith that is adapted to move between a first shape and a second shape during the process of forming an internal cavity within tissue. The process of forming the cavity may involve the flexible cutting element cutting, compressing, and otherwise affecting tissue as the shaft and/or an associated insertion tube are rotated about a longitudinal axis. It will be appreciated that the term “axis” shall mean a line being linear, substantially linear, straight, or curvilinear. Cavity formation may also be effectuated by impacting tissue or displacing tissue as the shaft is either partially or completely rotated, translated axially, or otherwise actuated. The internal cavity formed by the device may have a significantly larger diameter than the diameter of the initial opening used to insert the device into the tissue. Tissue cavities created in accordance with versions herein may be of any suitable size, shape, or configuration including a substantially spherical cavity, a substantially hemispherical cavity, a substantially linear cavity, a groove, a channel, a cavity having varying geometries, such as an upper hemispherical chamber and a lower linear cavity, or any other suitable cavity.

In numerous versions, the present invention comprises biasing, translating, or otherwise moving the flexible cutting element from a first shape to a second shape, or vice versa, such that a cutting element may be inserted through a relatively narrow hole and then opened to form a relatively large cavity. Methods of biasing the flexible cutting element include, but are not limited to, providing a spring bias arising from elastic and/or plastic deformation of the flexible cutting element, providing bias arising from a thermal shape-memory alloy, providing bias arising from centrifugal force generated as the shaft is rotated, providing bias arising from a tension cable that forcefully actuates a shape change, and providing bias from a coiled cutting element that projects laterally when uncoiled. The term “lateral” shall mean of, related to, moving in the direction of, moving substantially in the direction of, moving substantially in the direction away from, situated at, situated on, or situated at about the side of an element or situated or extending away from the medial plane of a body or element. The term “transverse” shall mean lying, extending, or projecting in a cross direction or lying across the axis of a part, element, or body. The term “cutting” shall mean to penetrate with as with a sharp edged instrument, to divide, to hew, to saw, to abridge, to shorten, to detach, to rub, to excavate, to hollow out, to divide into pieces, to excise, or to reduce.

Versions of the device may be operated by conventional surgical drills, with a conventional T-handle, a straight handle, a screw drive, knobs, slides, rotational members, levers, actuators, or with any other suitable device. In versions incorporating a T-handle, the T-handle also may be adapted to apply tension to the tension cable and/or to rotate a cable. It will be appreciated that devices used in accordance with versions herein may be used to apply compression and/or tension. Reference to the term “actuator” herein refers to any suitable control member, support member, base member, actuation member, handle member, drive member, and/or articulation member. It will be appreciated that the actuator or handle need not be configured for grasping with the human hand and that the handle may include any suitable device or mechanism for operation or support with robotics or otherwise

Versions of the flexible cutting element may eliminate the need for complex and expensive assemblies with numerous moving parts to effectuate lateral cutting. The shape-changing behavior of the flexible cutting element may enable the device to be adapted to a shape suitable for minimally invasive placement in tissue. Providing a variety of shapes, configurations, cutting surfaces, and/or positions for one or a plurality of cutting elements may offer a user a wide range of options from which to choose when forming or modifying a bone cavity. Additional concepts and examples will be disclosed in accordance with further examples described herein.

FIG. 2A shows one version of a cavitation device 100 attached to a surgical drill 12. In the illustrated version, the surgical drill 12 is battery powered and illustrates one possible method of operation. The surgical drill 12 may rotate at about, for example, 5,000 rotations per minute, or at any other suitable rotational speed. In versions incorporating the use of a drill, it may be beneficial to provide a cavitation device 100 that may be operated at rotational speeds lower than that of a 40,000-80,000 rpm drill such that excessive heat, vibration, and the potential for stressed or broken components or bone are reduced. Efficiently creating cavities at lower rotations per minute may increase the overall efficacy and safety of the procedure. There are myriad options for either powered or manual operation of cavitation device 100 that may be used in accordance with versions herein. For powered operation, the device may be used with a variety of readily available surgical drills that are pneumatic or electric. Alternatively, the shaft may be connected to any suitable mechanical actuator.

As shown in FIG. 2B, cavitation device 100 includes a shaft 110, a flexible cutting element 120, and a cutting tip 130. In the illustrated version, the shaft 110 has a longitudinal axis 111 and a generally circular cross-section. It will be appreciated that any suitable cross-section, such as a generally square cross-section, a generally elliptical cross-section, or a polygon cross-section are contemplated. In the illustrated version, the cavitation device 100 includes an insertion tube 114, having an aperture 124, where the flexible cutting element 120 is configured to be housed or retained at least partially within the insertion tube 114.

Still referring to FIG. 2B, in one version, the flexible cutting element 120 includes a free end 121 and has a relatively thin, rectangular, cross-section. Thus, the flexible cutting element 120, in one version, is consistent with a machine element known as a leaf spring and also is consistent with a structural element known as a cantilever beam. Because of this configuration, the flexible cutting element 120 may be deformably configured to transition between a first shape 122, in which the flexible cutting element 120 is substantially colinear with the longitudinal axis 111 of shaft 110, and a second shape 123, in which flexible cutting element 120 extends or projects away from longitudinal axis 111 laterally in the general shape of a curvilinear arc, as shown in FIG. 2B. The term “deform” shall mean to change the form of or transform, to alter the shape of or misshape, or to alter the shape of by pressure or stress.

As illustrated in FIG. 2B, the movement of the flexible cutting element 120 from a first shape 122 to a second shape 123 results in the free end 121 projecting laterally through the aperture 124. Subsequent rotation of the shaft 110 and/or the insertion tube 114 may rotate the flexible cutting element 120 clockwise or counterclockwise to form or modify a cavity in tissue or for any other suitable purpose. The terms “projecting,” “projectable,” and “projection” disclosed herein shall refer to thrusting, extending, opening, expanding, uncoiling, relaxing, and/or otherwise moving outward, forward, and/or away from a reference point.

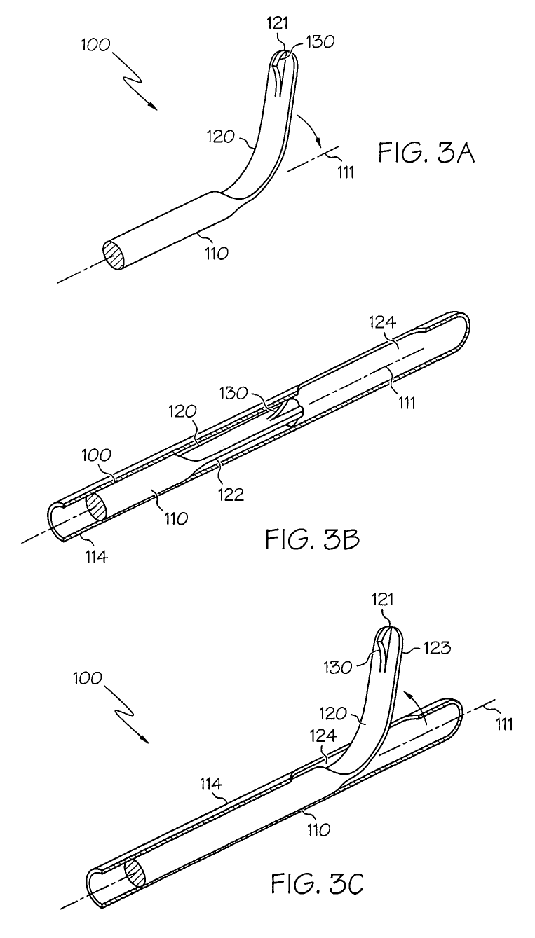

FIGS. 3A-3C illustrate an alternate version of the shape-changing behavior of cavitation device 100. As shown in the version illustrated in FIG. 3A, when flexible cutting element 120 is in its initial undeformed state the free end 121 extends or projects away from the longitudinal axis 111 of the shaft 110. However, as shown in FIG. 3B, the cavitation device 100 is dimensioned to pass through the interior of an insertion tube 114. The aperture 124 of the insertion tube 114 may be positioned at about the distal end of the insertion tube 114, may be elliptical in shape, and may be configured to allow the flexible cutting element 120 to be opened or otherwise extended therethrough. It will be appreciated that the aperture 124 may be configured with any suitable size, shape, configuration, and/or position. Depending on the particular surgical application, the insertion tube 114 may be a trochar, a cannula, a needle, a lumen, a tube fixed to a handle, a tube detachably coupled to an actuator, or any other suitable insertion device.

In one version, the flexible cutting element 120 experiences elastic deformation as it is placed within the insertion tube 114 and assumes the first shape 122, in which the flexible cutting element 120 is substantially colinear with the longitudinal axis 111. In the illustrated version, the cutting tip 130 helps keep the flexible cutting element 120 aligned within the insertion tube 114 as it is passed therethrough.

Referring now to FIG. 3C, as the flexible cutting element 120 extends past the leading edge of the aperture 124 of the insertion tube 114, a spring bias tends to move the flexible cutting element 120 from the first shape 122 toward the second shape 123. Consistent with spring mechanics, the flexible cutting element 120 seeks to return to the second shape 123 because it is a spring unloaded configuration. By reversing the insertion process, the flexible cutting element 120 may be returned to the first shape 122 for removal.

The flexible cutting element 120 may be constructed from a wide spectrum of materials, including surgical-grade stainless steel, capable of elastic behavior. Consistent with spring mechanics, the shape change of flexible cutting element 120 may operate within the elastic or plastic deformation range of the material. Another suitable material is the metal alloy Nitinol (NiTi), a biomaterial capable of superelastic mechanical behavior that can recover from significantly greater deformation relative to many other metal alloys. Alternatively, the flexible cutting element 120 may be constructed, for example, from a polymer, such as nylon or ultra high molecular weight polyethylene.

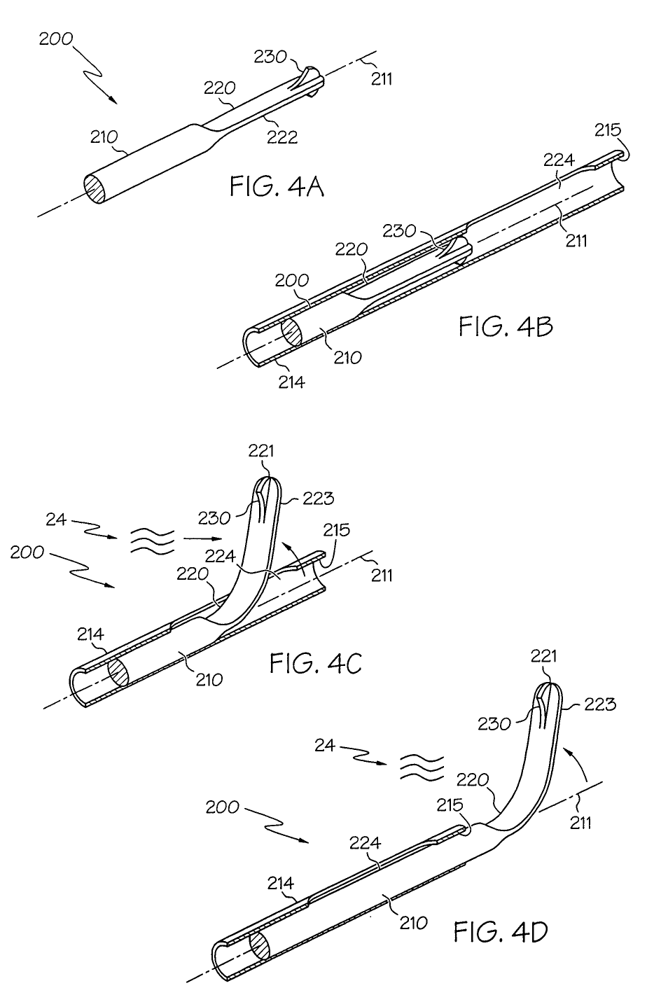

With reference, in particular, to FIGS. 4A-4D, Nitinol, or any other thermal shape-memory alloy, may also be used in accordance with a flexing device or method for biasing a flexible cutting element to move from a first shape to a second shape. For example, a flexible cutting element made from Nitinol may be deformed below a transformation temperature to a shape suitable for percutaneous placement into tissue. The reversal of deformation may be observed when the flexible cutting element is heated through the transformation temperature. The applied heat and/or cooling may be electrical, direct, indirect, from the surrounding tissue, and/or associated with frictional heat generated during operation.

FIGS. 4A-4D show an alternate version of a cavitation device 200, comprising a shaft 210 and a flexible cutting element 220 having a free end 221 and a cutting tip 230. The flexible cutting element 220 may be formed from a thermal shape-memory alloy, such as Nitinol, which is capable of shape change arising from thermal shape-memory behavior. In the illustrated version, the shaft 210 has a longitudinal axis 211.

FIG. 4A shows the cavitation device 200 in preparation for insertion, with the flexible cutting element 220 deformed below the transformation temperature to a first shape 222 in which the flexible cutting element 220 is substantially colinear with the longitudinal axis 211. When in the first shape 222, the flexible cutting element 220, retained within an insertion tube 214, may be passed through a pilot hole, or the like, into tissue.

Referring now to FIG. 4C, as the flexible cutting element 220 extends past the leading edge of the aperture 224 of the insertion tube 214, applied heat 24 activates the thermal shape-memory properties of the flexible cutting element 220. The applied heat and/or cooling may be electrical, direct, indirect, from the surrounding tissue, and/or associated with frictional heat generated during operation. The flexible cutting element 220 may have a bias toward a “remembered” second shape 223, in which the flexible cutting element 220 extends or projects away from the longitudinal axis 211 of the shaft 210 in the general shape of a curvilinear arc, as shown in FIG. 4C. Once in the second shape 223, the rotation shaft 210 and/or the insertion tube 214 may be rotated in a clockwise and/or counterclockwise direction to form or modify a tissue cavity.

Referring to FIG. 4D, an alternate configuration of the cavitation device 200 is disclosed. In the illustrated version, as the flexible cutting element 220 extends past the distal end 215 of the insertion tube 214, applied heat 24 activates the thermal shape-memory properties of flexible cutting element 220. Upon activation, the flexible cutting element 220 may be converted into the second shape 223. In the illustrated version of FIG. 4D, the shaft 210 may be freely rotatable within a substantially static insertion tube 214.

With reference to FIGS. 4A-4D, and all other suitable versions disclosed herein, it may be advantageous to add additional features to enhance the performance of cavitation devices of versions disclosed herein and to enhance the process of cavity creation, cavity modification, and/or tissue removal. Numerous secondary features to aid in tissue cutting include serrated edges, threads, cutting flutes, protrusions, tips, barbs, protuberances, abrasive surfaces, and beveled edges on one or both sides of the cutting element. Variations and different combinations are possible without departing from the spirit of the present invention.

Geometric variations, within the spirit of the present invention, may be developed to enhance or alter the performance of the dynamic shape behavior. Examples of such variations include the cross-sectional shape and the length of a flexible cutting element. For example, as will be discussed in more detail herein, the cross-sectional shape of the flexible cutting element can form a quadrilateral such that the edges formed from the acute angles of the quadrilateral are adapted to aid in cutting. Further, the curvature of a flexible cutting element in the extended position may take a specific shape, where the shape of the tissue cavity need not be limited to combinations of cylindrical or hemispherical tissue cavities. Different tissue cavity shapes may be desirable for interfacing with an implant or to create a region for synthetic bone, bone paste, PMMA, bone matrix, bone cement, and/or other structural elements to match complex anatomical structures. Cavities may additionally be filled with balloons, therapeutic agents, structural agents, dye agents, or left empty. In addition, a plurality of flexible cutting elements may be used, rather than only a single flexible cutting element, to achieve a desired result.

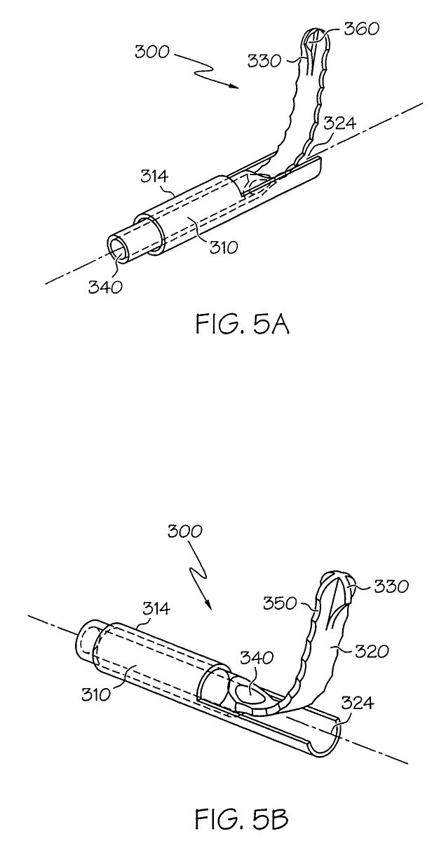

Referring now to FIGS. 5A-5B, one version of a cavitation device 300 includes a shaft 310 and a flexible cutting element 320 having serrations 350 to aid in tissue cutting. Similarly, a cutting tip 330 may comprise a cutting flute 360 to aid in tissue cutting. The cavitation device 300 may also include an irrigation passage 340, which may serve as a conduit for tissue irrigation, for removal of bone tissue, for delivery of a filling material such as bone matrix, for the delivery of a structural material, and/or for any other suitable purpose. In the illustrated version, the cavitation device 300 includes a rotatable insertion tube 314 having an aperture 324 therein. In the illustrated version, the aperture 324 is a combination of a distal aperture and a lateral aperture. The combined distal and lateral aperture may provide a user with flexibility as to where the flexible cutting element 320 is laterally and/or axially extended. The combined aperture 324 may offer a user with a wide range of cutting options. It will be appreciated that the lateral aperture portion of the aperture 324 may extend longitudinally for any suitable length and may otherwise be suitably configured.

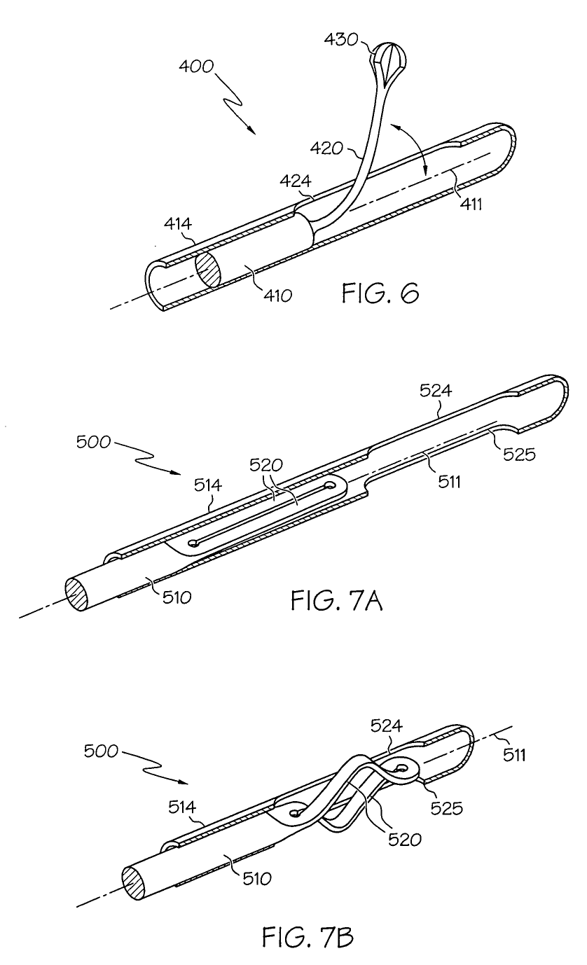

FIG. 6 depicts an alternate version of a cavitation device 400 including a shaft 410 having longitudinal axis 411. The cavitation device 400 further includes a flexible cutting element 420, having a club-shaped end 430, and a rotatable insertion tube 414 having an aperture 424. In one version, the aperture 424 is configured for the flexible cutting element 420 to extend therethrough.

Referring to FIG. 7A, an alternate version of a cavitation device 500 is depicted having a shaft 510 and a plurality of flexible cutting elements 520. In the illustrated version, the plurality of flexible cutting elements 520 are retained within an insertion tube 514 having a first aperture 524 and a second aperture 525 formed therein to accommodate the plurality of flexible cutting elements 520. FIG. 7A shows the cavitation device 500 with the flexible cutting elements 520 substantially colinear with longitudinal axis 511 of the shaft 510, consistent with a first shape suitable for minimally invasive placement within tissue. Referring now to FIG. 7B, the flexible cutting elements 520 are shown in a second shape, where portions of the flexible cutting elements 520 extend laterally or project away from longitudinal axis 511 through the first aperture 524 and the second aperture 525, respectively.

It will be appreciated that in the illustrated version of FIGS. 7A-7B the flexible cutting elements 520 form a closed loop that may be configured to take a desirable specific shape. The insertion tube 514 may be provided with any suitable number of apertures 524 to facilitate the lateral projection of one or a plurality of flexible cutting elements 520. The illustrated version of the flexible cutting elements 520 is disclosed by way of example only, where a plurality of flexible cutting elements of any suitable configuration may project from the insertion tube 514 laterally or axially.

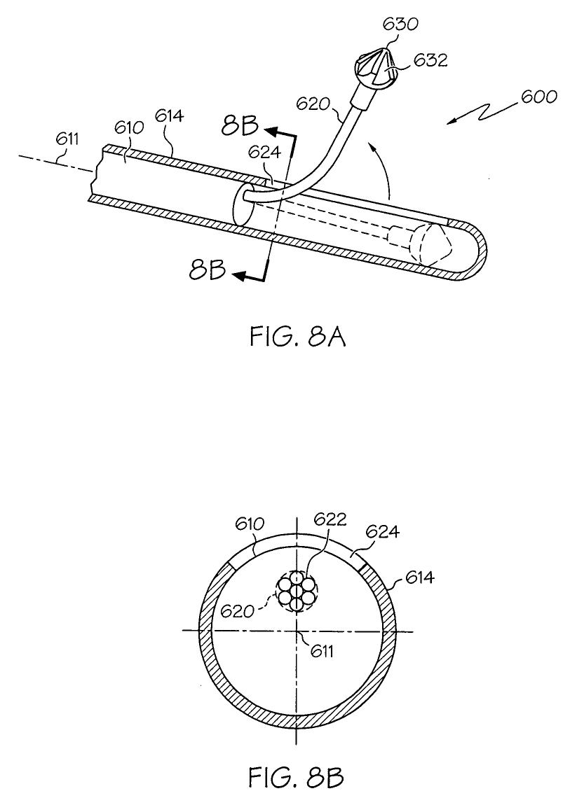

Another flexing method for biasing a flexible cutting element to move from a first shape toward a second shape utilizes centrifugal force arising from rotational velocity of the shaft. Centrifugal force is the force that tends to impel a thing or parts of a thing outward from a center of rotation. FIG. 8A shows an alternate version of a cavitation device 600 comprising a shaft 610 with longitudinal axis 611 and a flexible cutting element 620 having a cutting tip 630 and cutting flutes 632, where the cavitation device 600 is housed or at least partially retained within an insertion tube 614 having an aperture 624 therein.

In the illustrated version, the flexible cutting element 620 has a generally circular cross-section. FIG. 8B shows the cross-section of flexible cutting element 620, taken along line 8B-8B, as having a standard cable structure with a uniform helical arrangement of wires 622 concentrically stranded together. This type of cable structure may provide high strength and high flexibility.

Still referring to FIG. 8B, the flexible cutting element 620 is shown offset from the longitudinal axis 611 to further encourage outward movement of the flexible cutting element 620 under the influence of centrifugal forces that arise when the shaft 610 and the insertion tube 614 are rotated at sufficient velocity. The cavitation device 600 may be driven by a surgical drill capable of rotational velocity greater than about 5,000 revolutions per minute, or by any other suitable device.

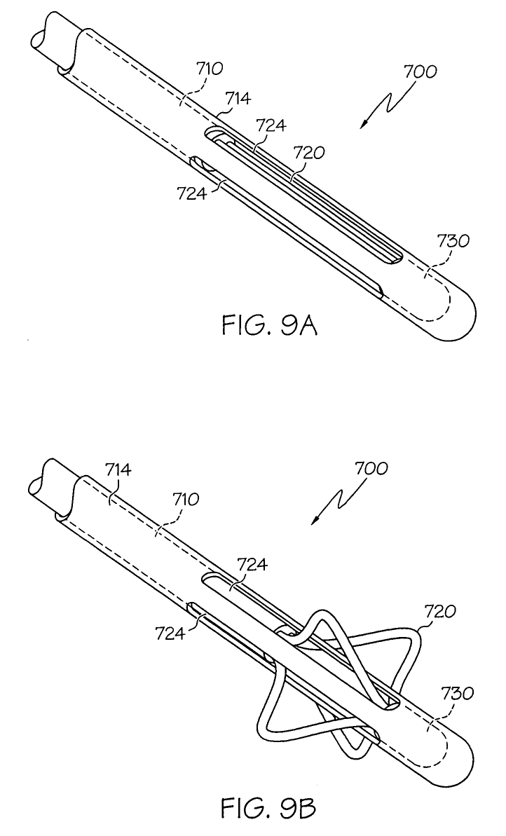

Referring to FIGS. 9A-9B, an alternate embodiment of a cavitation device 700 is shown. Referring to FIG. 9A, a plurality of flexible cutting elements 720 are generally colinear with a shaft 710 to form a first shape suitable for minimally invasive placement of the device within tissue or into a pilot hole. In one version, the proximal ends of the flexible cutting elements 720 are rigidly attached to the shaft 710 and the distal ends of the flexible cutting elements 720 are attached to a spindle 730. In the illustrated version, the flexible cutting elements 720 shown in the first shape are housed at least partially within an insertion tube 714 having a plurality of apertures 724 corresponding to the plurality of flexible cutting elements 720. Providing a plurality of cutting elements may improve the speed and efficiency of cavity creation. Providing a plurality of cutting elements, particularly if the cutting elements are of different configurations, may also allow portions of a cavity having varying geometries to be created simultaneously.

Referring now to FIG. 9B, when the cavitation device 700 and the insertion tube 714 are rotated at a sufficient rotational velocity, the flexible cutting elements 720 have a tendency to bow outward under the influence of centrifugal force. Additionally or independently, the operator may advance the shaft 710 toward spindle 730 to assist in moving the flexible cutting elements 720 from the first shape toward a second shape, in which the flexible cutting elements extend outwardly from the axis of rotation. With reference to this and other versions, although the distal end of the insertion tube 714 is shown sealed, it will be appreciated that the distal end may have an open configuration such that the cavitation device 700 may be extended therethrough. The version illustrated with reference to FIGS. 9A-9B may be operated with the benefit of centrifugal force, manual actuation, or both.

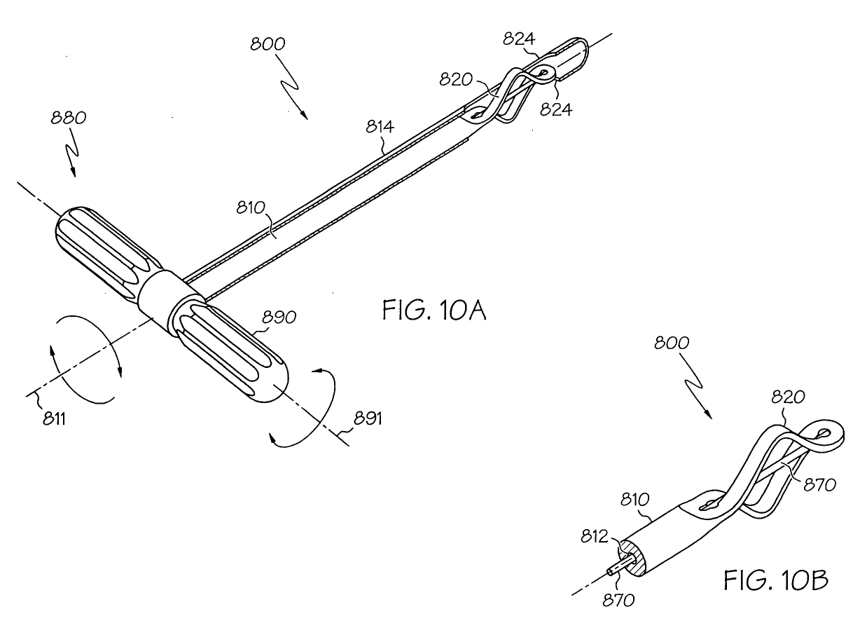

FIGS. 10A-10B depict an alternate embodiment of a cavitation device 800 comprising a shaft 810 having longitudinal axis 811 and flexible cutting elements 820 housed or maintained at least partially within a rotatable insertion tube 814 having a plurality of apertures 824 therein. The rotatable insertion tube 814 may be coupled, permanently or detachably, to a T-handle 880 such that, during a procedure, rotation of the T-handle 880 correspondingly rotates the insertion tube 814. In one version, upon completion of the procedure, the insertion tube 814 may be unscrewed or otherwise disconnected from the T-handle 880.

In the illustrated version, the shaft 810 additionally has a control passage 812 running substantially along the longitudinal axis 811. In the illustrated version, a tension cable 870 is connected to the flexible cutting elements 820 and extends proximally through the control passage 812. The proximal end of cavitation device 810 is attached to the T-handle 880 having a grip 890, with the proximal end of tension cable 870 being attached to grip 890 such that rotation of grip 890 about its longitudinal axis 891 applies a tension force to tension cable 870. Thus, the tension cable 870 is a flexing mechanism or device for biasing the flexible cutting elements 820 to move from a first shape toward a second shape. As the grip 890 is rotated about its longitudinal axis 891, tension is applied to tension cable 870, thereby applying compressive and bending forces to flexible cutting elements 820 and causing them to extend outward toward a second shape. The T-handle 880 may also be rotated about longitudinal axis 811 to form a tissue cavity. It will be appreciated that any other suitable actuator, such as a straight handle, a drill, a knob, a lever, or the like may be provided in accordance with versions herein.

Referring to FIGS. 11A-11C, one version of a method for cavity 48 formation is shown where the periphery of the target tissue, such as bone, is accessed with a guide member 106 placed percutaneously. In particular, FIGS. 11A-11C are directed to forming a cavity 48 in osteoporotic cancellous bone followed by filling of the cavity with a strengthening synthetic bone that is injectable and hardens in vivo. This method is generally applicable to all means for shape change behavior of the flexible cutting elements described herein.

Referring to FIG. 11A, in one version, a standard surgical drill and drill bit are used to create a pilot hole 46 in bone through a guide member 106 using established techniques. It will be appreciated that the pilot hole 46 may be created with a surgical drill, an electric drill, a manual drill, by manually pushing or urging a component, with a punch, with suction, or by any other suitable method. It is contemplated that the pilot hole may be any suitable shape or configuration including, for example, a track in which a cavitation device may slide, a cross-shape, a cylindrical hole wide enough to allow a cavitation device to pivot about the edge of the hole, or the like. The bone structure shown in FIG. 11A includes cortical bone 44 and cancellous bone 42. A flexible cutting element 120 of cavitation device 100, shown in FIG. 11A, is in a first shape adapted for passage to the distal end of pilot hole 46. In the illustrated version, the cutting tip 130 helps to keep the flexible cutting element 120 centered within the insertion member 114 during passage through the guide tube 106 and pilot hole 46. Once placed, the shaft 110 and/or insertion tube 114 may be used to transmit torsion to the flexible cutting element 120.

Referring now to FIG. 11B, as the shaft 110 and the insertion tube 114 rotate about the axis A-A, the flexible cutting element 120 moves toward a second shape during the process of forming a generally hemispherical tissue cavity 48 with a cavity height 50. As illustrated, the diameter of cavity 48 is twice the size of cavity height 50. It will be appreciated that any suitable cavity shape, size, or configuration may be created, as will be illustrated in more detail herein where, for example, a cylindrical or partially cylindrical cavity may be created with a degree of rotation from 0 degrees to 360 degrees about the longitudinal axis A-A at any suitable distance from the longitudinal axis A-A.

FIG. 11C shows the tissue cavity 48 being filled with, for example, an injectable material 16, such as synthetic bone, that hardens in vivo. Prior to being filled with a synthetic bone, or the like, the tissue cavity 48 may be cleared with suction or irrigation. Any suitable filler, bonding, structural, or therapeutic agent may be administered into the cavity. For example, polymethylmethacrylate (PMMA), commonly referred to as bone cement, is a well-known bone synthetic substitute that may be injected or inserted into a bone cavity. Other synthetic bone substitutes include resorbable and non-resorbable materials such as injectable calcium phosphate and injectable terpolymer resin with combeite glass-ceramic reinforcing particles. Filling materials may include structural agents, therapeutic agents, dye agents, inflatable elements, bone paste, bone matrix, synthetic matrix, growth agents such as hydroxyapatite, and/or any other suitable material. It will be appreciated that an inflatable device may be inserted into a cavity to stabilize or otherwise assist in healing a fractured bone. Cavities may also be unfilled.

Osteoporosis can be a contributing factor to fractures of bone, especially of the femur, radius, humerus, and vertebral bodies. There are several non-invasive methods for determining bone mineral density, and patients at high risk for fracture can be identified. Patients with previous fractures related to osteoporosis are at high risk for re-fracture or initial fractures of other bone structures. Minimally invasive devices and methods, such as those describe herein, combined with synthetic bone substitutes, may provide for the strengthening of bone, a preventive treatment for patients at high risk of fracture.

Bone may be removed through known irrigation and suction methods. In the case of bone harvesting, the abated bone is used at another surgical site to promote healing of a bony deficit or to promote joint fusion. The cavity may then be filled with a suitable bone substitute, such as a synthetic matrix, that is injectable and hardens in vivo. When removing and replacing osteoporotic bone, the cavity may be filled with structural synthetic bone or bone cement. Since the device and methods of the present invention are generally minimally invasive, they may be used for the prevention of osteoporosis related fractures in individuals at high risk. Skeletal structures where osteoporosis related fractures are common include the radius, femur, and vertebral bodies.

According to an alternate version, the periphery of the target tissue, such as bone, may be accessed with an insertion tube placed percutaneously, and a pilot hole may be formed in the bone with a standard surgical drill and drill bit or by any other suitable insertion device or mechanism including pushing a pilot hole forming element into the bone. Next, the device of the present invention may be inserted to a suitable depth within the pilot hole. The flexible cutting element of the device may then be moved from a first shape to a second shape such that the cutting element extends laterally through an aperture in the insertion tube. Portions of the cutting element extend away from the longitudinal axis of the shaft into contact with bone tissue such that upon rotation, or other suitable movement, a tissue cavity is formed or modified.

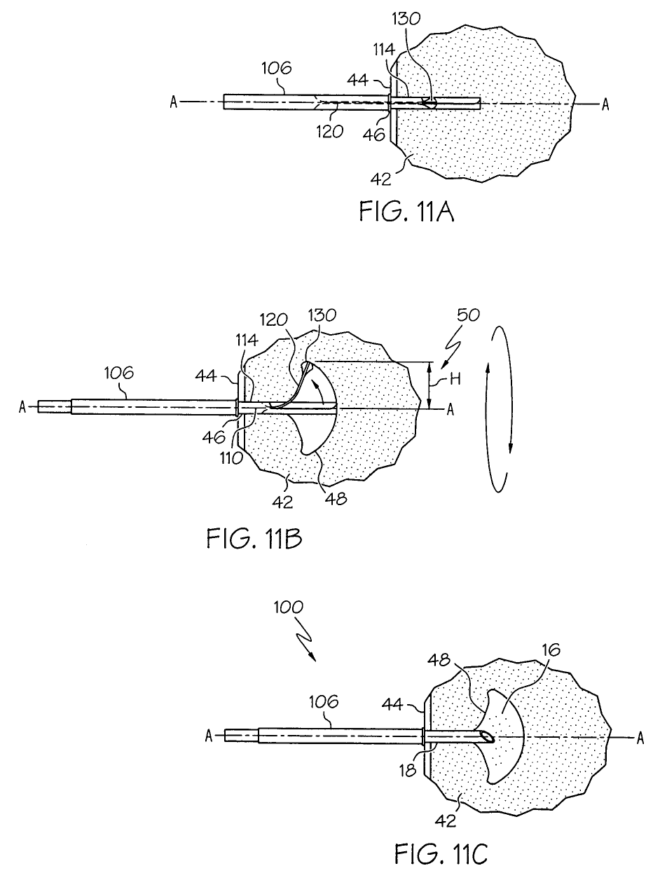

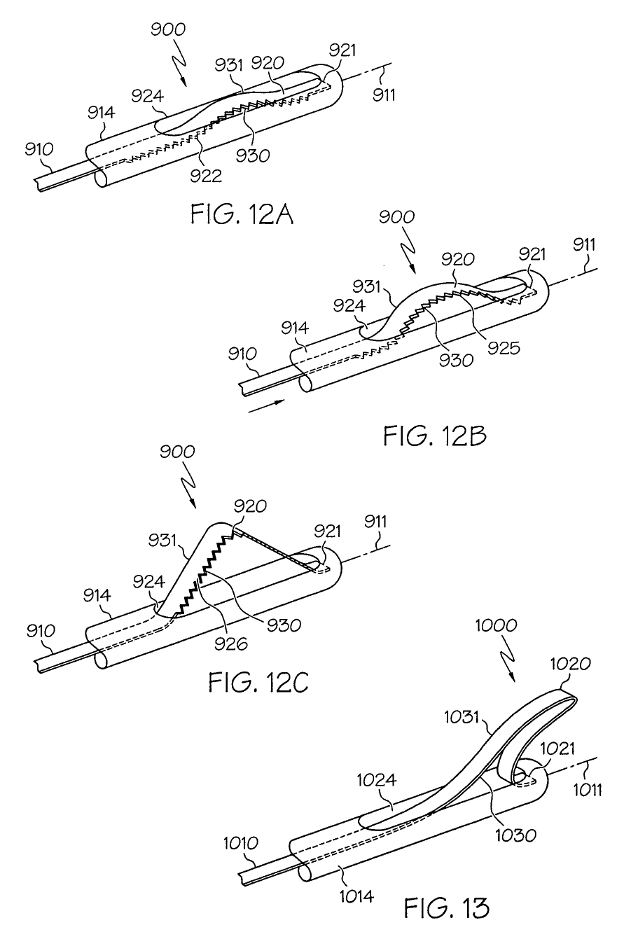

FIGS. 12A-12C show an alternate version of a cavitation device 900 comprising a shaft 910 formed integrally with or coupled with a flexible cutting element 920 having a fixed distal end 921, a first cutting edge 930, and a second cutting edge 931. In the illustrated version, the cavitation device 900 is housed or partially retained within an insertion tube 914 having an aperture 924 therein. In the illustrated version, the flexible cutting element 920 is formed from a flexible material, such as stainless steel. The shaft 910 has a longitudinal axis 911.

FIG. 12A shows the cavitation device 900 with the flexible cutting element 920 configured in a first shape 922 in which the flexible cutting element 920 is aligned generally adjacent the longitudinal axis 911 such that it is retained within the insertion tube 914. The flexible cutting element 920 may be constructed from any suitable material including, for example, stainless steel or Nitinol. When the flexible cutting element 920 is in the first shape 922, the cavitation device 900 may be inserted into a pilot hole in accordance with a minimally invasive procedure. The flexible cutting element 920 may be provided with a guide element, such as a guide ridge having a slight bend, as illustrated, such that it is biased towards opening through the aperture 924 and away from the axis 911. It will be appreciated that any other suitable method of encouraging the flexible cutting element 920 to open through the aperture 924 is contemplated.

Referring now to FIG. 12B, in one version, as the shaft 910 is compressed, or otherwise urged distally, the flexible cutting element 920 opens through the aperture 924 of insertion tube 914 to form a second shape 925. The flexible cutting element 920 may be compressed or otherwise moved from the first shape 922, shown in FIG. 12A, to one or a plurality of cutting shapes by any suitable articulation method, such as with a T-handle. For example, in one version, the proximal end of the shaft 910 is attached to a T-handle, such as T-handle 880 of FIG. 10A, having a grip 890. The proximal end of the shaft 910 may be attached to the grip 890 such that rotation of the grip 890 about its longitudinal axis applies a compression force to the shaft 910. Thus, the shaft 910 is a flexing mechanism or device for biasing the flexible cutting element 920 to move from a first shape 922 toward a second shape 925 or toward any suitable number of shapes. As the grip 890 is rotated about its longitudinal axis, compression is applied to the shaft 910, thereby applying compressive and bending forces to the flexible cutting element 920, causing it to extend outward toward a second shape. The T-handle 880, as applied to all versions herein, may then be rotated manually about longitudinal axis 911 to form or modify a tissue cavity.

In one version, the flexible cutting element 920 is generally colinear with and/or adjacent the longitudinal axis 911 when configured in a first shape 922, where compression applied by proximally actuating the insertion tube 914 moves the flexible cutting element 920 from a first shape 922 to a second shape 925. In an alternate version, the flexible cutting element 920 has a bias toward a “remembered” second shape 925, in which the flexible cutting element 920 extends or projects away from longitudinal axis 911 of the shaft 910 in the general shape of a curvilinear arc, as shown in FIG. 12B. The actuator, T-handle, or the like, may be used to apply tension to the shaft 910 such that the flexible cutting element is actively drawn into the first shape 922 shown in FIG. 12A. Releasing the tension on the shaft 910 allows the flexible cutting element 920 to return to its resting or second shape 925.

Referring to FIG. 12C, the flexible cutting element 920 may be moved to a third shape 926 in accordance with versions herein such as, for example, by compressing the shaft 910. The flexible cutting element 920 may be urged or otherwise moved from a first shape to one or a plurality of cutting shapes by any suitable articulation method such as with a T-handle, manual actuator, or electrical actuator. The third shape 926 may, for example, project laterally outward farther from the longitudinal axis 911 than the second shape 925, shown in FIG. 12B. Providing a plurality of available cutting shapes with one flexible cutting element 920 may increase the number of options available to a physician forming or modifying tissue cavities.

Upon opening, the flexible cutting element 920 may extend or project away from the longitudinal axis 911 of the shaft 910 in the general shape of a curvilinear arc or in any other suitable shape. The memory retention aspects of a number of materials, such as Nitinol or stainless steel, allow for a wide range of possible configurations that may be provided. Various shapes may also be provided by, for example, varying hardness, varying material, varying response to temperature, and varying flexibility at different regions of the flexible cutting element.

The first shape 922, the second shape 925, and the third shape 926 may be selected prior to a procedure or during a procedure. For example, a first cavity may be created with the flexible cutting element 920 configured in the second shape 925. After completion of the first cavity, the flexible cutting element 920 may be changed into the third shape 926 to increase the size of the first cavity to create a second cavity. It is contemplated that a user may alternate between shapes, configurations, and directions while creating a cavity without removing the cavitation device from the bone. Shapes may be pre-set such that a user may select a predictable shape from a selection such that the user knows precisely which shape is being used to cut tissue. It will be appreciated that the first shape 922, the second shape 925, and the third shape 926 may be discreetly selectable configurations or, in an alternate version, may be points along a continuum that may be selected during or prior to a procedure. Providing a plurality of selectable configurations and/or allowing a user to adjust the cutting element may allow for precise cavity creation or modification.

Versions of the flexible cutting element may be configured, articulated, or manipulated into any suitable shape such as, for example, an arcuate shape, a plateau shape, a curvilinear shape, a coiled shape, a helical shape, a laterally extended shape, a convex shape, a concave shape, a linear shape, and/or a sinusoidal or wave-shape. The shaft portion may be integral and contiguous with the flexible cutting element or may be a more clearly defined or discreet actuation member coupled with the flexible cutting element. The distal end of the flexible cutting element may be permanently fixed to an insertion tube or a cap member such that the distal end remains static as the shaft is tensioned, rotated, compressed, articulated, and/or otherwise moved to change the flexible cutting element from a first shape to a second shape. As will be further discussed herein, in alternate versions, the distal end of the flexible cutting element may be freely movable within an insertion tube and/or may be detachably coupled to the insertion tube.

FIG. 13 depicts an alternate version of a cavitation device 1000 comprising a shaft 1010 and a flexible cutting element 1020 having a fixed distal end 1021, a first cutting edge 1030, and a second cutting edge 1031. In the illustrated version, the cavitation device 1000 includes an insertion tube 1014 having an aperture 1024 therein. In the illustrated version, the flexible cutting element 1020 is formed from a flexible material, such as Nitinol, which is capable of shape change and shape-memory behavior. The shaft 1010 has a longitudinal axis 1011 about which the flexible cutting element 1020 may be rotated to form or modify a cavity. In particular, FIG. 13 illustrates an alternate shape of the flexible cutting element 1020, where a portion of the flexible cutting element 1020 projects distally beyond the end of the insertion tube 1014. The illustrated version of the cavitation device 1000 may be used to form or modify cavities both laterally and distally situated with respect to the distal end of the insertion tube 1014.

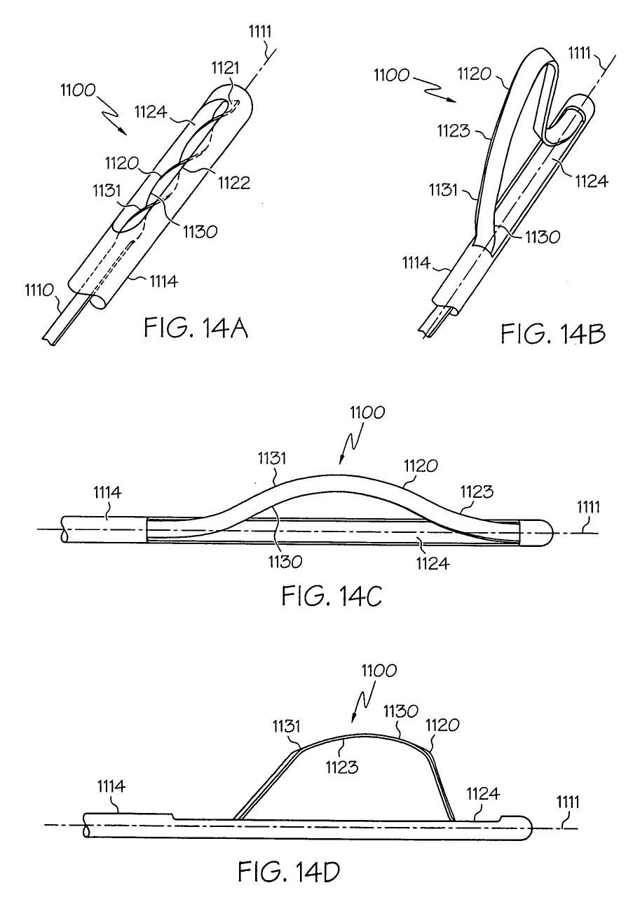

Referring to FIGS. 14A-14D, an alternate version of a cavitation device 1100 is shown comprising a shaft 1110 and a flexible cutting element 1120 having a fixed distal end 1121, a first cutting edge 1130, and a second cutting edge 1131. In the illustrated version, the flexible cutting element 1120 is housed or partially retained within an insertion tube 1114 having an aperture 1124 therein. The flexible cutting element 1120 is formed from a flexible material, such as Nitinol, which is capable of shape change and shape-memory behavior. The shaft 1110 has a longitudinal axis 1111.

FIG. 14A shows the cavitation device 1100 coiled into a substantially helical first shape 1122 in which the flexible cutting element 1120 is coiled or wound such that it is substantially aligned with the longitudinal axis 1111 and is housed within the insertion tube 1114. When the flexible cutting element 1120 is in the first shape 1122, the cavitation device 1100 may be passed through a pilot hole in accordance with a minimially invasive procedure. The first shape 1122 may be effectuated with an applied torque or, alternatively, may be a remembered shape that may be unwound with an applied torque into an uncoiled shape. The term “helical” shall mean pertaining to or having the form of a spiral, helix, and/or coil.

Referring now to FIG. 14B, in the illustrated version, as the shaft 1110 is unwound or untwisted, the flexible cutting element 1120 extends laterally through the aperture 1124 of the insertion tube 1114 into a second shape 1123. The flexible cutting element 1120 may be untwisted, or otherwise moved, from the first shape 1122 to one or a plurality of cutting shapes by any suitable articulation method or device. For example, in one version, the proximal end of the shaft 1110 is attached to a T-handle, such as T-handle 880 of FIG. 10A, having a grip 890, with the proximal end of the shaft 1110 being attached to the grip 890 such that rotation of the grip 890 about its longitudinal axis 891 rotates the shaft 1110. Thus, in one version, the shaft 1110 is a rotation device for torsioning the flexible cutting element 1120 to move from a first shape 1122, shown in FIG. 14A, toward a second shape 1123, or toward any suitable number of shapes, due to the applied torque from an actuator. As the grip 890 is rotated about its longitudinal axis 891 the flexible cutting element 1120 may unwind, thereby causing it to extend outward laterally toward a second shape 1123. Once the flexible cutting element 1120 has assumed the second shape 1123, the T-handle 880, or the like, may be rotated manually about the longitudinal axis 1111 to form a tissue cavity. It will be appreciated that disclosed methods of operation may be applied to all versions of the cavitation device disclosed herein.

Referring to FIGS. 14A-14D, the flexible cutting element 1120 may be constructed from Nitinol, or any other suitable memory retention material, where upon uncoiling the shaft 1110 the flexible cutting element 1120 resumes the second shape 1123. As illustrated, the second shape 1123 may project outward through the aperture 1124 in an arcuate shape that is offset from the longitudinal axis 1111. As it uncoils, the flexible cutting element 1120 may project laterally at a slope such that the flexible cutting element 1120 is offset from the aperture 1124. The offset configuration of the second shape 1123, shown in particularity with reference to the top view of FIG. 14C, may make the coiled first shape 1122 the most efficacious way to retain the cavitation device 1100 within the insertion tube 1114 for a minimially invasive procedure.

Referring, in particular, to FIG. 14C, the second shape 1123 of the flexible cutting element 1120 comprises a concave first cutting edge 1130 and a convex second cutting edge 1131. As applies universally to all versions herein, the shaft 1110, as shown in FIG. 14A, and/or the insertion tube 1114, may be rotated in a clockwise and/or counterclockwise direction to form or modify a desired cavity. Providing a first cutting edge 1130 with a concave surface and a second cutting edge 1131 with a convex surface may provide the user with desirable options and transition cut geometries for cavity formation. Providing non-linear cutting geometries may provide an effective longitudinal cutting edge that is graduated or tapered. Varying the rotational direction of the shaft 1110 may allow a user to cut or push through tissue with the convex point of the second cutting edge 1131 and/or cut or pull through tissue with the concave trough of the first cutting edge 1130. It will be appreciated that any dimension or degree of convexity or concavity may be applied to all or a portion of the cutting edges 1130, 1131 of the flexible cutting element 1120. It is further contemplated that the body of the flexible cutting element 1120 may be provided with a plurality of lateral concave and convex portions, with reference to the longitudinal axis 1111, where the convexities and concavities may create, for example, a wave-like appearance in the flexible cutting element 1120.

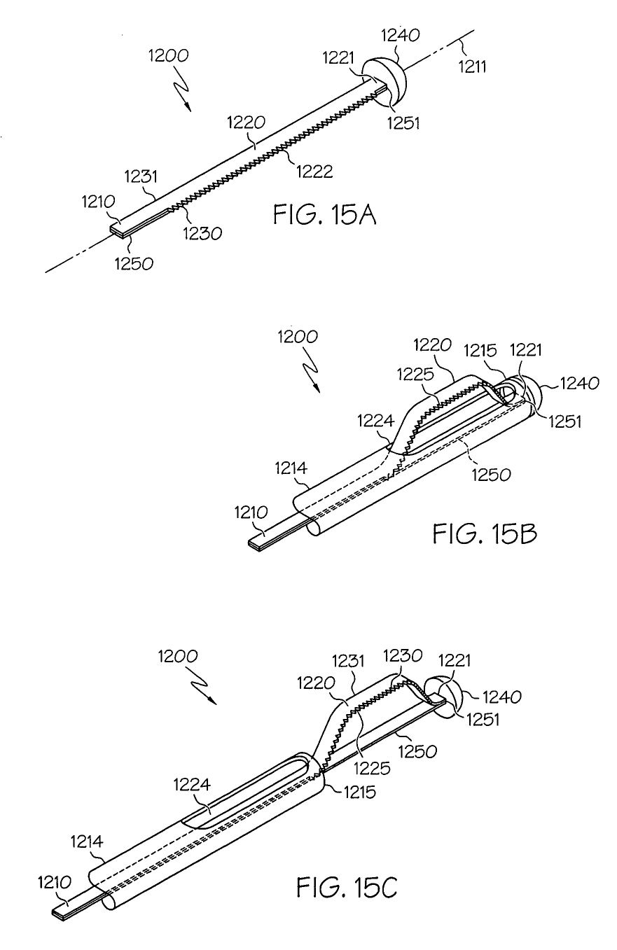

FIGS. 15A-15C depict an alternate version of a cavitation device 1200 comprising a shaft 1210 associated with a flexible cutting element 1220 having a fixed distal end 1221 attached to a tip 1240, a first cutting edge 1230, and a second cutting edge 1231. In the illustrated version, the tip 1240 is not permanently fixed to the distal end 1215 of the insertion tube 1214 such that it may translate longitudinally about the longitudinal axis 1211. Providing flexibility in the positioning of the flexible cutting element 1220 may provide a user with a variety of cavity formation options. Configured to operate in cooperation with the flexible cutting element 1220 is a support member 1250 having a fixed end 1251 attached to the tip 1240. In the illustrated version, the flexible cutting element 1220 and the support member 1250 may be housed or partially retained within an insertion tube 1214 having an aperture 1224 therein. The flexible cutting element 1220 may be formed from a flexible material, such as Nitinol, which is capable of shape change and shape-memory behavior. The support member 1250 may be constructed from a rigid or semi-rigid material.

FIG. 15A depicts the flexible cutting element 1220 in a first shape 1222, where the flexible cutting element 1220 is positioned generally colinear with the longitudinal axis 1211 and is adjacent the support member 1250. When the flexible cutting element 1220 is in the first shape 1222, the cavitation device 1200 may be passed into a pilot hole, or otherwise inserted into tissue, in accordance with a minimally invasive procedure. The flexible cutting element 1220 and the support member 1250 may be fixed, detachably coupled, freely movable, and/or otherwise suitably configured in association with an insertion tube 1214, shown in FIGS. 15B-15C. It will be appreciated that the flexible cutting element 1220 and the support member 1250 may be operated independently from the insertion tube 1214.

Referring now to FIG. 15B, in one version, as the shaft 1210 is urged distally the flexible cutting element 1220 is pushed against the tip 1240 such that the flexible cutting element 1220 extends laterally through the aperture 1224 of the insertion tube 1214 to form a second shape 1225. When the flexible cutting element 1220 is converted into the second shape 1225, for example, by distally urging the shaft 1210, the support member 1250, fixed to the tip 1240, may be tensioned or drawn proximally to provide an opposite force to allow the flexible cutting element 1220 to open. It will be appreciated that any other suitable method of operation is contemplated, such as where the shaft 1210 is held static and the tip 1240 is drawn proximally with the support member 1250 to convert the first shape 1222 into the second shape 1225. Once open, the shaft 1210, the support member 1250, and/or the insertion tube 1214 may be rotated to cut tissue.

Referring to FIG. 15C, in one version, the support member 1250 may be used to push the tip 1240 distally outward from the open end 1215 of the insertion tube 1214 such that the flexible cutting element 1220 is distal to the end 1215. As illustrated, after being extended, the flexible cutting element 1220 may be configured into the second shape 1225 by distally urging the shaft 1210 and simultaneously tensioning or drawing the support member 1250 in the opposite direction. FIG. 15C illustrates one version, by way of example only, of an alternate method of opening a flexible cutting element 1220.

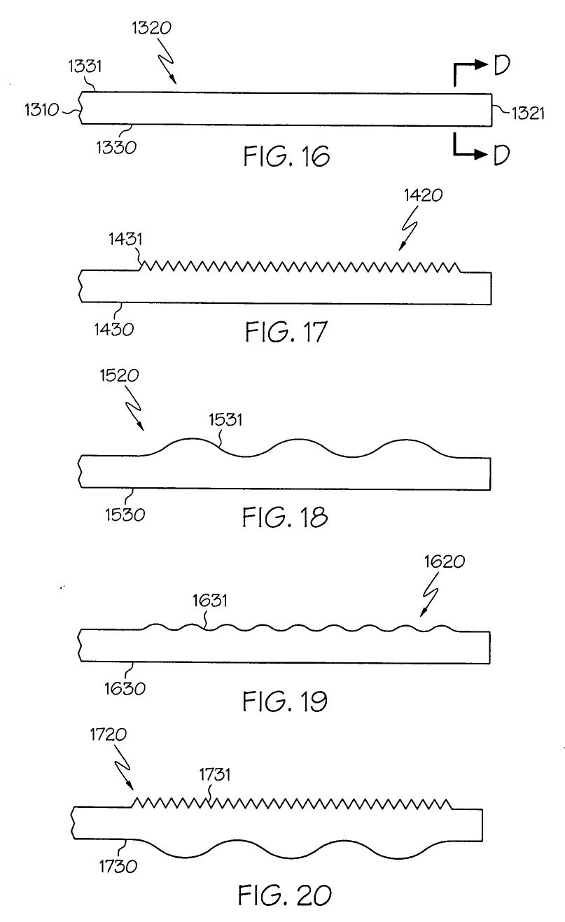

Although any suitable shape or configuration is contemplated, FIGS. 16-20 illustrate various longitudinal cutting edges or surface effects for flexible cutting elements in accordance with versions herein. The one or a plurality of flexible cutting elements may be rotated in a clockwise and/or counterclockwise direction to form or modify a cavity. In addition to being rotatable or movable in one or a plurality of directions, the flexible cutting elements may be provided with one or a plurality of surface effects to create different cutting effects. Multiple cutting edges or surface effects may be combined in a single flexible cutting element to affect tissue differently depending upon the direction of cut. The term “surface effect” shall refer to any geometry, feature, projection, texture, treatment, edging, sharpening, tapering, material type, hardness, memory retention, heat treating, response to heat, roughness, smoothness, sharpness, shape, and/or configuration of one or a plurality of surfaces, faces, edges, points, or the like, of the flexible cutting element or any other component of a cavitation device.

Referring to FIG. 16, one version of a flexible cutting element 1320 is shown having a first cutting edge 1330, a second cutting edge 1331, a distal end 1321, and a shaft portion 1310. In the illustrated version, the flexible cutting element 1320 is a longitudinally extending member configured to rotationally cut through tissue. The first cutting edge 1330 and the second cutting edge 1331 may be provided with a surface effect for cutting or modifying tissue in a clockwise and/or counterclockwise direction. In the illustrated version, the first cutting edge 1330 and the second cutting edge 1331 are substantially smooth and planar such that the same cutting effect will be achieved in both the clockwise and counterclockwise direction. Referring to FIGS. 16-20, surface effects refer to the texture, configuration, shape, or the like, of one or a plurality of cutting surfaces or edges of a flexible cutting element that are operably configured to cut or modify tissue. Any suitable surface effect is contemplated including, but not limited to, serrations, waves, convexities, concavities, edging, points, sharpened edges, smooth edges, rounded edges, flat edges, hardened edges, or combinations thereof. It is further contemplated that a first surface effect may be provided on a first cutting surface and a second surface effect may be provided on a second cutting surface of a flexible cutting element such that varying the direction of rotation varies the type of cut or tissue effect.

For example, FIG. 17 depicts one version of a flexible cutting element 1420 having a smooth first cutting edge 1430 and a serrated second cutting edge 1431. In use, the user may alternate between cutting with the smooth first cutting edge 1430 and the serrated second cutting edge 1431 to produce the desired tissue effect. FIG. 18 depicts one version of a flexible cutting element 1520 having a smooth first cutting edge 1530 and a wavy second cutting edge 1531. FIG. 19 depicts one version of a flexible cutting element 1620 having a smooth first cutting edge 1630 and an alternate version of a wavy second cutting edge 1631. FIG. 20 depicts one version of a flexible cutting element 1720 having a wavy first cutting edge 1730 and a serrated second cutting edge 1731. The illustrated surface effects are disclosed by way of example and are not intended to be limiting. Surface effects disclosed herein, including variations and combinations thereof, may be incorporated into any suitable flexible cutting element.

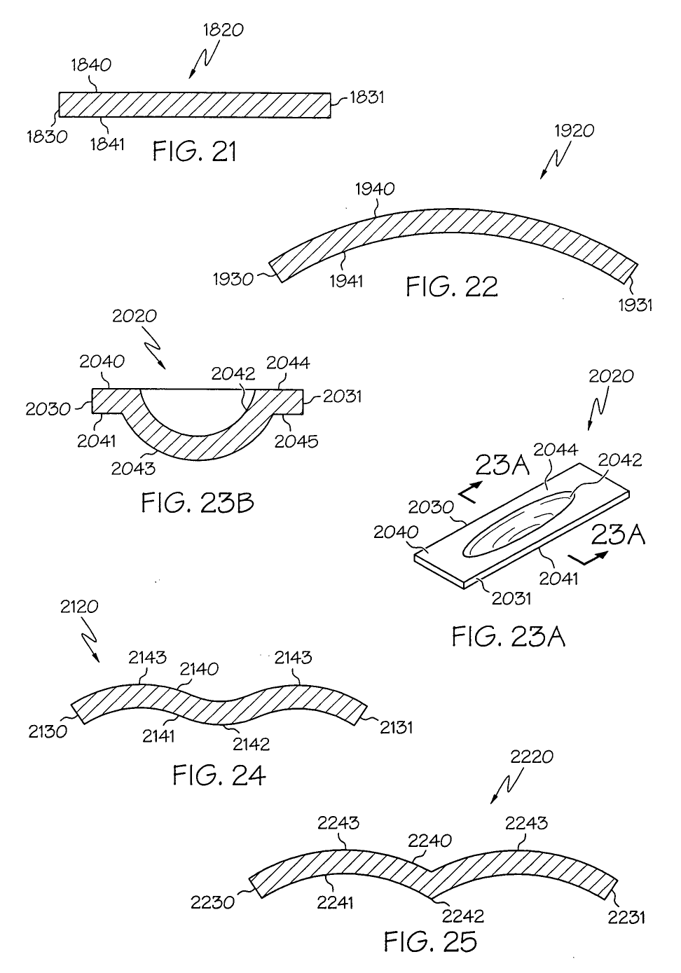

FIGS. 21-40 refer, generally, to examples of lateral cross-sections of a flexible cutting element taken along axes corresponding to reference line D-D of FIG. 16. Any suitable cross-section may be provided, where altering the shape, size, and/or configuration of the flexible element may advantageously alter the cutting effect, the stiffness, the sharpness, and/or other properties of the flexible cutting element. It will be appreciated that the illustrated versions are disclosed by way of example only and are not intended to be limiting where, for example, illustrated configurations may be combined with other illustrated configurations wholly or partially.

FIG. 21 illustrates one version of a lateral cross-section of a flexible cutting element 1820 having a first cutting edge 1830, a second cutting edge 1831, a top surface 1840, and a bottom surface 1841. The cross-section view may be taken, for example, along line D-D illustrated in FIG. 16. In the illustrated version, the first cutting edge 1830 and the second cutting edge 1831 are parallel planar surfaces, and the top surface 1840 and the bottom surface 1841 are parallel planar surfaces forming a parallelogram. The cross-section illustrated in FIG. 21, as applies to all cross-sections disclosed herein and variations thereof, may be for all or a portion of the flexible cutting element and/or may change shape during use. For example, the cross-section of FIG. 21 may be the cross-section of a portion of the flexible cutting element 1120 in the second shape 1123, as illustrated in FIGS. 14B-14D. In the first shape 1122, shown in FIG. 14A, the cross-section of the wound flexible cutting element 1120 may be dramatically different. The illustrated cross-sections are disclosed by way of example only to illustrate numerous options that may be available to users to achieve a desired tissue effect.

FIG. 22 illustrates one version of a lateral cross-section of a flexible cutting element 1920 having a first cutting edge 1930, a second cutting edge 1931, a top surface 1940, and a bottom surface 1941. In the illustrated version the top surface 1940 is convex and the bottom surface 1941 is concave. Providing one or a plurality of convexities and concavities may alter the cutting angle and cutting effect of the flexible cutting element 1920 on tissue. Additionally, the concavities and/or convexities positioned may improve the strength or rigidity of the flexible cutting element. An angled or curved cutting edge may permit a more gradual cut of tissue that requires less force to complete.

FIGS. 23A-23B illustrate one version of a flexible cutting element 2020 having a first cutting edge 2030, a second cutting edge 2031, a top surface 2040, and a bottom surface 2041. Referring to FIG. 23A, illustrated is a perspective view of a portion of the flexible cutting element 2020, where a portion of the top surface 2040 includes a tapered concavity 2042. Providing the flexible cutting element 2020 with the concavity 2042 may improve the strength, rigidity, and/or cutting effect of the flexible cutting element, for example, at a particular point of weakness or stress. Referring to FIG. 23B, in the illustrated version, the bottom surface 2041 includes a convexity 2043 corresponding in size and shape to the tapered concavity 2042 such that the thickness of the flexible cutting element 2020 is substantially constant along the length and width thereof. It will be appreciated that convexities and/or concavities may be of varying shape, thickness, and configuration from one another and the flexible cutting element 2020. The top surface 2040 may further include a substantially planar top portion 2044 and the bottom surface 2041 may include a substantially planar bottom portion 2045, where the concavity 2042 functions as a rib or bridge between the planar portions 2044, 2045 of the flexible cutting element 2020. It will be appreciated that any suitable number of concavities and/or convexities may be provided having any suitable shape or configuration.

As illustrated in FIGS. 23A-23B, the flexible cutting element 2020 may have a first lateral cross-section at a first region and a differing second lateral cross-section at a second region. For example, in the region of the concavity 2042, the flexible cutting element 2020 may have the cross-section illustrated in FIG. 23B. In planar regions, the flexible cutting element 2020 may have a cross-section similar to the cross-section illustrated in FIG. 21. Varying the cross-sections of the flexible cutting element 2020 along the length thereof may provide advantageous tissue effects and/or may be structurally advantageous. It will be appreciated that any suitable variation or alternation in cross-section is contemplated where, for example, versions disclosed herein may be used in combination.

FIG. 24 illustrates one version of a lateral cross-section of a flexible cutting element 2120 having a first cutting edge 2130, a second cutting edge 2131, a top surface 2140, and a bottom surface 2141. The top surface 2140 and the bottom surface 2141 may have one or a plurality of convexities and/or concavities configured such that the lateral cross-section has a wave-like or sinusoidal configuration. In the illustrated version, the flexible cutting element 2120 includes a central peak 2142 and two outer peaks 2143 that may be advantageous structurally or for tissue formation. The central peak 2142 may function for structural support as a rib or spine along the central axis of the flexible cutting element 2120. The outer peaks 2143 may provide support and/or an angled cutting surface. The peaks 2142, 2143 may be beveled, rounded, or otherwise suitably shaped.

FIG. 25 illustrates one version of a lateral cross-section of a flexible cutting element 2220 having a first cutting edge 2230, a second cutting edge 2231, a top surface 2240, and a bottom surface 2241. The top surface 2240 and the bottom surface 2241 may have one or a plurality of convexities and/or concavities configured such that the lateral cross-section has a wave-like or sinusoidal configuration. In the illustrated version, the flexible cutting element 2220 includes a central peak 2242 and two outer peaks 2243 that may be advantageous structurally or for tissue formation. The central peak 2242 may function for structural support as a rib or spine along the central axis of the flexible cutting element 2220. The outer peaks 2243 may provide support and/or an angled cutting surface. The peaks 2242, 2243 may be beveled, rounded, angled, pointed, ridged, or otherwise suitably shaped.

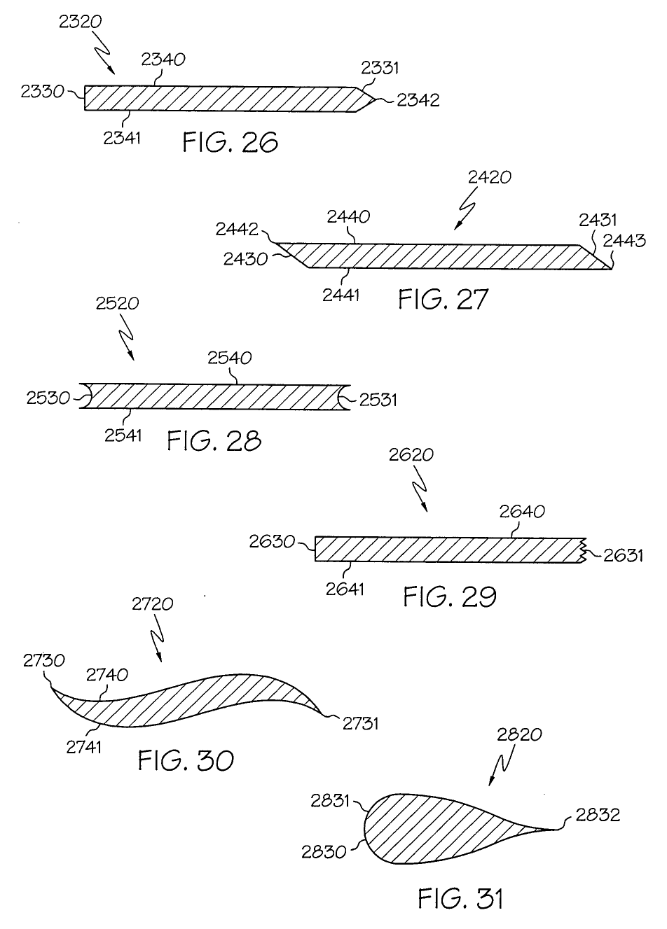

FIG. 26 illustrates one version of a lateral cross-section of a flexible cutting element 2320 having a first cutting edge 2330, a second cutting edge 2331, a top surface 2340, and a bottom surface 2341. In the illustrated version, the first cutting edge 2330 is substantially planar and perpendicular to the top surface 2340 and the bottom surface 2341. The second cutting edge 2331 tapers to a tip 2342. Providing a first cutting edge 2330 and a second cutting edge 2331 with different surface geometries provide a user with multiple options to choose from when forming or modifying a cavity where, for example, the desired cutting edge 2330, 2331 may be selected by the direction of rotation.

FIG. 27 illustrates one version of a lateral cross-section of a flexible cutting element 2420 having a first cutting edge 2430, a second cutting edge 2431, a top surface 2440, and a bottom surface 2441. In the illustrated version, the first cutting edge 2430 is substantially parallel to the second cutting edge 2431, and the top surface 2440 is substantially parallel to the bottom surface 2441 to form a parallelogram. The first cutting edge 2430 includes a first wedge-shaped tip 2442 and the second cutting edge 2431 includes a second wedge-shaped tip 2443 facing in substantially opposite directions. Providing a first cutting edge 2430 and a second cutting edge 2431 that differ from one another may provide a user with multiple options to choose from when forming or modifying a cavity, where the desired cutting edge 2430, 2431 may be selected by the direction of rotation.

FIG. 28 illustrates one version of a lateral cross-section of a flexible cutting element 2520 having a first cutting edge 2530, a second cutting edge 2531, a top surface 2540, and a bottom surface 2541. In the illustrated version, the first cutting edge 2530 and the second cutting edge 2531 are concave to create cutting points at the intersection with the top surface 2540 and the bottom surface 2541. FIG. 29 illustrates one version of a lateral cross-section of a flexible cutting element 2620 having a first cutting edge 2630, a second cutting edge 2631, a top surface 2640, and a bottom surface 2641. In the illustrated version the second cutting edge 2631 is serrated.

FIG. 30 illustrates one version of a lateral cross-section of a flexible cutting element 2720 having a first surface 2740 and a second surface 2741. The surfaces of the flexible cutting element 2720 may be tapered such that they intersect at a first cutting tip 2730 and a second cutting tip 2731. The surfaces 2740, 2741 may be wave-shaped, contain convexities and/or concavities, contain tapers, and/or any other suitable geometry. FIG. 31 illustrates one version of a lateral cross-section of a flexible cutting element 2820 having a cutting surface 2830, where the flexible cutting element 2820 is configured with an acute point 2832 and a rounded end 2831.

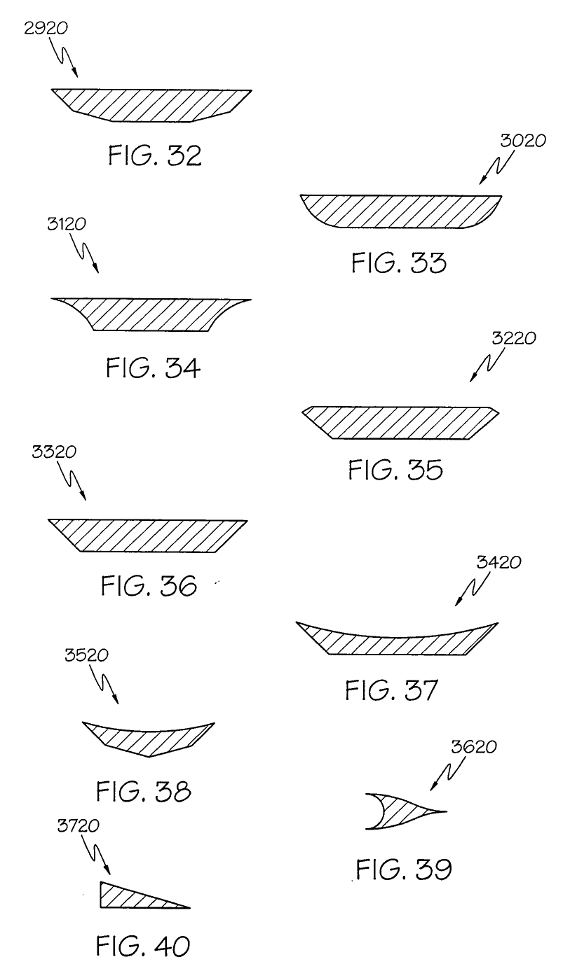

It will be appreciated that versions of the flexible cutting element may have any suitable lateral cross-section configuration. For example, FIGS. 32-40 illustrate additional configurations of flexible cutting elements 2920, 3020, 3120, 3220, 3320, 3420, 3520, 3620, and 3720. FIGS. 32-40 disclose versions of lateral cross-sections taken along a reference line corresponding to line D-D illustrated in FIG. 16.

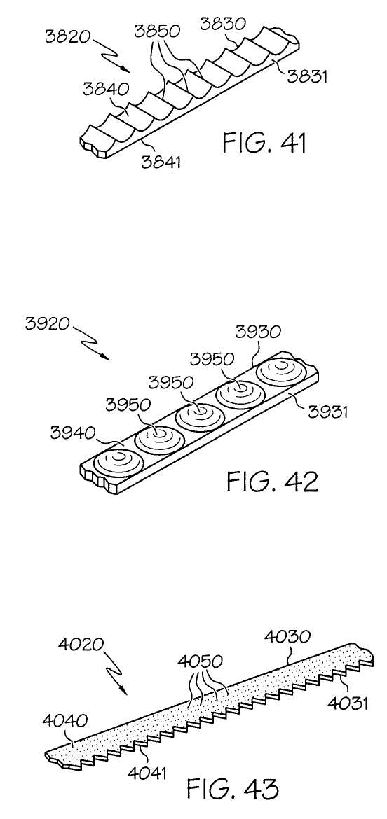

FIG. 41 illustrates one version of a portion of a flexible cutting element 3820 operably configured to form or modify tissue cavities with axial motion in addition to rotation. The flexible cutting element 3820 includes a first cutting edge 3830, a second cutting edge 3831, a top surface 3840, and a bottom surface 3841. In the illustrated version, the first cutting edge 3830 and the second cutting edge 3831 may be rotated clockwise or counter clockwise, as discussed previously, to form or modify tissue cavities. Additionally, the top surface 3840 of the flexible cutting element 3820 may be provided with one or a plurality of cutting elements 3850 configured to cut tissue when the flexible cutting element 3820 is repeatedly opened and closed with axial motion.

For example, the top surface 3840 of the flexible cutting element 3820 may include cutting elements 3850 in the form of ridges that may be used with an axial or sawing motion to create a lateral cavity such as that illustrated in FIG. 48. The lateral cavity of FIG. 48 may be formed without rotation by using solely axial motion. A first and second method for cavity formation absent rotational motion are disclosed.

In a first method, a cavitation device, such as the cavitation device 900 of FIGS. 12A-12C, may be provided with a top surface having one or a plurality of cutting elements 3850. For example, by repeatedly alternating the cavitation device 900 between the first shape 922 to the second shape 925, as illustrated in FIGS. 12-A and 12-B, the cutting element 3850 combined therewith may laterally cut into bone tissue with a sawing motion to create a cavity.

In a second method, the cavitation device 900 having one or a plurality of cutting elements 3850 may be opened laterally until the cutting element elements 3850 are adjacent bone tissue. The instrument may then be translated in an axial or sawing motion, with the flexible cutting element 3820 in a static position, to create a cavity. If a larger cavity is desired, the cutting elements 3850 may be extended laterally until contact is again made with bone tissue. The cavitation device 900 may then, as before, be translated in an axial or sawing motion. In this manner the cavitation device may be used in accordance with a stepping method to create a desirable cavity.

In a third method, the cavitation device 900 having one or a plurality of cutting elements 3850 may be opened laterally until the cutting element elements 3850 are adjacent bone tissue. The instrument, or a cutting portion thereof, may then be rotated to create a cavity. If a larger cavity is desired, the cutting elements 3850 may be extended laterally until contact is again made with bone tissue. The cavitation device 900 may then be rotated again to create a larger cavity. In this manner the cavitation device may be used in accordance with a rotational stepping method to create a desirable cavity.

The lateral cutting functionality of the flexible cutting element 3820 may be used in combination with rotational cutting to form or modify tissue cavities. For example, once a cavitation device, such as the cavitation device 900 of FIGS. 12A-12C, is inserted into a pilot hole, the flexible cutting element may initially be used to create a lateral cavity, such as the tissue cavity depicted in FIG. 48. The flexible cutting element may then be extended laterally into the lateral cavity and rotated in a clockwise or counterclockwise direction to cut tissue. The initial lateral cavity may provide an advantageous starting point from which rotational cavity formation may originate.

FIG. 42 illustrates an alternate version of a portion of a flexible cutting element 3920 operably configured to form or modify tissue cavities with rotational and/or axial motion. The flexible cutting element 3920 includes a first cutting edge 3930, a second cutting edge 3931, a top surface 3940, and a bottom surface 3941. In the illustrated version, the first cutting edge 3930 and the second cutting edge 3931 may be rotated clockwise or counter clockwise, as discussed herein, to form or modify tissue cavities. Additionally, the top surface 3940 of the flexible cutting element 3920 may be provided with one or a plurality of cutting elements 3950 that may cut tissue when the flexible cutting element 3920 is repeatedly opened and closed with axial motion. In the illustrated version, the cutting elements 3950 are convex bumps that may be textured to provide a cutting surface for forming lateral cavities.

FIG. 43 illustrates an alternate version of a portion of a textured flexible cutting element 4020 operably configured to form or modify tissue cavities with rotational and/or axial motion. The flexible cutting element 4020 includes a first cutting edge 4030, a second cutting edge 4031, a top surface 4040, and a bottom surface 4041. In the illustrated version, the first cutting edge 4030 and the second cutting edge 4031 may be rotated clockwise or counter clockwise, as discussed herein, to form or modify tissue cavities. Additionally, the top surface 4040 of the flexible cutting element 4020 may be provided with a plurality of cutting elements 4050 that may cut or erode tissue abrasively when the flexible cutting element 4020 is repeatedly opened and closed with axial motion. In the illustrated version, the cutting elements 4050 are surface effects creating texture on the top surface 4040. The texture may be created with added particular matter, with small machined projections, by scoring the top surface 4040, or by any other suitable method. It will be appreciated that the one or a plurality of cutting elements may be any surface effect, device, configuration, sharpness, and/or additive configured to aid the flexible cutting element in forming a lateral cavity with axial motion. The one or a plurality of cutting elements may also alter the tissue effect when the flexible cutting element is rotated such as, for example, with projecting lateral ridges.

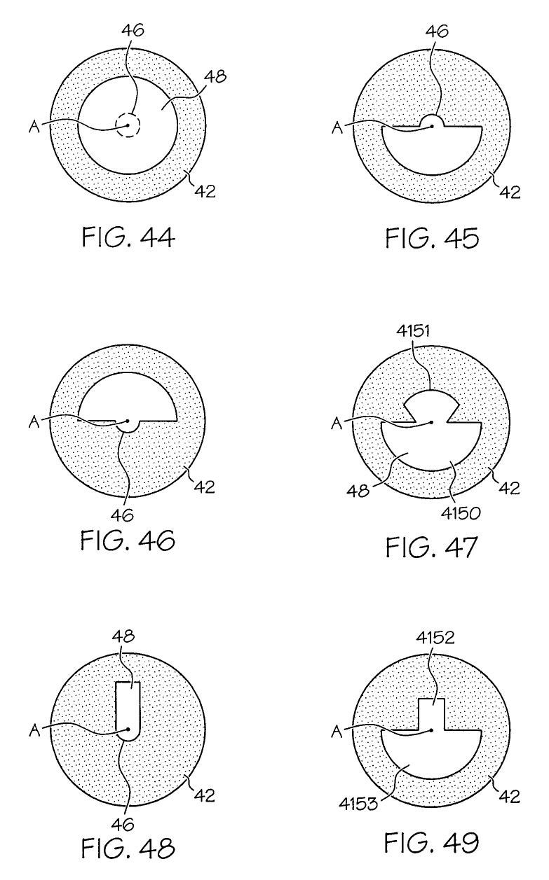

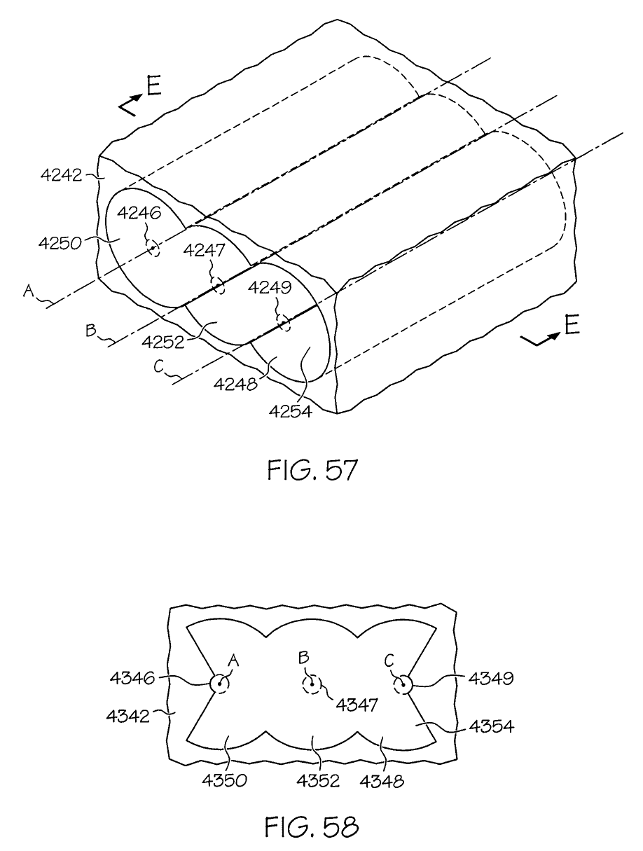

FIGS. 44-62 illustrate examples of cross-sections of tissue cavities 48 formed in accordance with versions herein. The cross-sections may, for example, be views of different versions of tissue cavities 48 taken along axes corresponding to the axis A-A shown in FIGS. 11A-11C. As discussed with reference to FIGS. 11A-11C, a pilot hole 46 may be formed in, for example, cancellous bone tissue 42 for the insertion of a cavitation device, such as the cavitation device 900 of FIGS. 12A-12C. Upon insertion, the cavitation device may be changed, for example, from a first shape configured for insertion into a second shape configured to form or modify tissue cavities 48. The tissue cavities 48 may be formed with clockwise rotational cutting, counterclockwise rotational cutting, lateral cutting, and/or by any other suitable cutting method or device. The cross-sections of tissue cavities disclosed herein may be formed or modified with any suitable cavitation device in accordance with versions herein. It will be appreciated that the tissue cavities 48 are disclosed by way of example and are not intended to be limiting. It will be appreciated that the tissue cavities may be provided in any suitable tissue and that versions depicted herein are disclosed by way of example only.

FIG. 44 illustrates one version of a tissue cavity 48 that may be formed, for example, by inserting a cavitation device, such as the cavitation device 900 of FIGS. 12A-12C, into a pre-formed pilot hole 46 with an axis A-A and then rotating the cavitation device about the axis A-A 360 degrees. In the illustrated version, the pilot hole 46 is visible only as phantom lines as the rotation of the cavitation device about the axis A-A expands the pilot hole 46 into the tissue cavity 48. As applies to all versions here, tissue cavities 48 may be created for any suitable number of reasons including, for example, the treatment or prevention of bone fracture, joint fusion, implant fixation, tissue harvesting, removal of diseased tissue (hard or soft tissue), general tissue removal (hard or soft tissue), vertebroplasty, and kyphoplasty. The tissue cavities 48, upon formation or modification, may be filled with therapeutic agents, structural materials, devices, inflatable members, fluids, gasses, and/or any other suitable material, including combinations thereof. It will be appreciated that the tissue cavities 48 may also be left empty.

FIGS. 45-46 illustrate versions of a tissue cavity 48 that may be formed by inserting a cavitation device into a pilot hole 46 and then rotating the cavitation device about the axis A-A 180 degrees. Cavitation devices in accordance with versions herein, such as the cavitation device 900 of FIGS. 12A-12C, may be configured to cut cavities of less than 360 degrees. Tissue cavities 48 in the shape of hemispheres may, for example, have structural or therapeutic benefits. Cavitation devices in accordance with versions herein may be used to create tissue cavities 48 within any suitable range of motion about the axis A-A. Configuring cavitation devices to provide cavities of less than 360 degrees may give a user flexibility in determining how best to treat a particular tissue region. Tailored tissue cavities 48 may have both therapeutic and structural benefits due to the precision in configuration that may be achieved.

Referring to FIG. 47, one version of a tissue cavity 48 is shown having a first tissue cavity region 4150 that may be formed in the same manner, for example, as the tissue cavity 48 of FIG. 45. The tissue cavity 48 of FIG. 47 includes a second tissue cavity region 4151 differently dimensioned than the first tissue cavity region 4150. In one version, the illustrated tissue cavity 48 of FIG. 47 may be formed with a cavitation device, such as the cavitation device 900 of FIGS. 12A-12C, having a single flexible cutting element 920. For example, after creating the first tissue cavity region 4150, the cavitation device may be rotated 180 degrees such that the cavitation device is now facing the opposite direction. The flexible cutting element of the cavitation device may then be used to create the second tissue cavity region 4151. In such a manner, multiple tissue cavity regions 4150, 4151 may be created having different dimensions, configurations, shapes, and/or sizes. Although a first tissue cavity region 4150 and a second tissue cavity region 4151 are disclosed, it will be appreciated that any suitable number of tissue cavity regions of any suitable configuration may be formed.

FIG. 48 illustrates one version of a tissue cavity 48 including a portion of a pilot hole 46. In the illustrated version, the tissue cavity 48 is a lateral tissue cavity that may be formed, for example, in accordance with the use of the flexible cutting element 3820 of FIG. 41. For example, the pilot hole 46 may initially be formed by drilling into bone tissue. A cavitation device, such as the cavitation device 900 of FIGS. 12A-12C having a flexible cutting element, such as the flexible cutting element 3820 of FIG. 41, may be inserted into the pilot hole 46. Once inserted, the flexible cutting element 3820, having a cutting element 3850 associated therewith, may be translated in a sawing motion to create a laterally projecting tissue cavity 48. Providing a cavitation device configured to form a lateral tissue cavity, such as the tissue cavity 48 illustrated in FIG. 48, may provide users an additional option to choose from when forming tissue cavities.

Referring to FIG. 49, one version of the tissue cavity 48 may include a lateral cavity portion 4152 that may be formed, for example, in the manner illustrated with reference to FIG. 48. The lateral tissue cavity portion 4152 may be combined with a second tissue cavity portion 4153 formed using rotational cutting in accordance with the description of FIGS. 45-46. FIG. 49 illustrates one version in which lateral cutting and rotation cutting may be combined to form a suitable cavity 48. It will be appreciated that by using, for example, the flexible cutting element 3820 of FIG. 41, that a single flexible cutting element may be configured for both cutting functions.

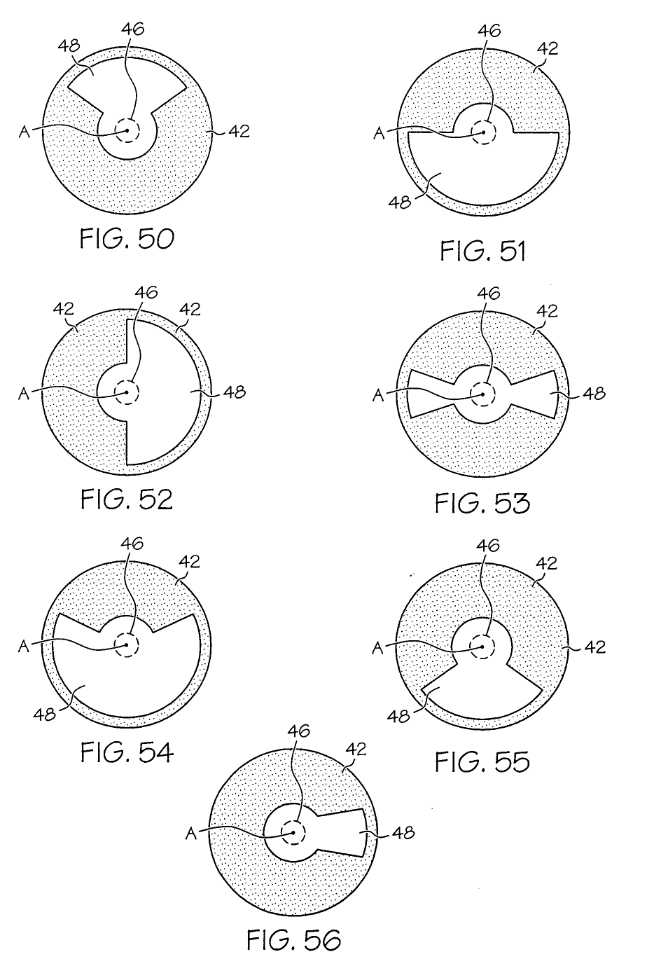

FIGS. 50-56 illustrate additional examples of tissue cavities 48 taken along axis A-A. FIGS. 50-56 illustrate that the pilot hole 46 having a first radius may be widened to a second radius, with reference to the central axis A-A, using a rotational cutting device. A second tissue cavity portion, of less than 360 degrees, may be created having a third radius greater than the second radius of the widened pilot hole 46. In this manner, multiple variations of tissue cavities 48 may be achieved having cavity portions with different radii with reference to the axis A-A. Tissue cavities disclosed herein are by way of example only, where it is contemplated that a plurality of tissue cavity variations may be provided in accordance with versions herein.

FIG. 57 illustrates one version of a tissue cavity 4248 configured by combining a first cavity portion 4250, a second cavity portion 4252, and a third cavity portion 4254. The first cavity portion 4250 may be formed, for example, in accordance with the description referencing the cylindrical cavity 48 formed in FIG. 44. The first cavity portion 4250 may be formed by inserting a cavitation device into a first pilot hole 4246 that may, for example, be pre-drilled into tissue. The cavitation device may then be rotated about an axis A-A in accordance, for example, with the description referencing FIGS. 12A-12C, to form the first cavity portion 4250. The second cavity portion 4252 may be formed by inserting a cavitation device into a second pilot hole 4247. The cavitation device may then be rotated about an axis B-B to form the second cavity portion 4252. The axes A-A and B-B may be oriented such that rotation of the cavitation device thereabout will create overlapping cavity portions. The third cavity portion 4254 may be formed by inserting a cavitation device into a third pilot hole 4249. The cavitation device may then be rotated about an axis C-C to form the third cavity portion 4254. The axes B-B and C-C may be oriented such that rotation of the cavitation device thereabout will create overlapping cavity portions. By operating the cavitation device in the disclosed manner, the three cavity portions 4250, 4252, 4254 may be formed such that they overlap to create a single tissue cavity 4248. It will be appreciated that the position of the axes is variable and is disclosed by way of example only, where the axes may be, for example, parallel, converging, overlapping, multiplanar, linear, non-linear, or the like.

Providing a plurality of connected tissue cavity portions may offer a user a wide range of options to choose from when designing a tissue cavity. The tissue cavity 4248 of FIG. 57 is disclosed by way of example, where it will be appreciated that any suitable number of cavity portions having any suitable number of configurations may be combined to form a desirable cavity. Tissue cavities created in the disclosed manner may be particularly well suited for receiving structural materials, such as PMMA, or for housing inflatable devices in accordance with kyphoplasty procedures.

FIG. 58 illustrates an alternate version of a tissue cavity 4348 taken along reference line E-E configured by combining a first cavity portion 4350, a second cavity portion 4352, and a third cavity portion 4354. The first cavity portion 4350 may be formed, for example, in accordance with the description referencing the cylindrical cavity 48 formed in FIGS. 45-46. The first cavity portion 4350 may be formed by inserting a cavitation device into a first pilot hole 4346 that may be, for example, pre-drilled. The cavitation device may then be rotated about an axis A-A in accordance with, for example, the description referencing FIGS. 12A-12C, to form the first hemispherical cavity portion 4350. The second cavity portion 4352 may be formed by inserting a cavitation device into a second pilot hole 4347. The cavitation device may then be rotated about an axis B-B to form the second cylindrical cavity portion 4352. The axes A-A and B-B may be oriented such that rotation of the cavitation device thereabout will create overlapping cavity portions. The third cavity portion 4354 may be formed by inserting a cavitation device into a third pilot hole 4349. The cavitation device may then be rotated about an axis C-C to form the third hemispherical cavity portion 4354. The axes B-B and C-C may be oriented such that rotation of the cavitation device thereabout will create overlapping cavity portions. By operating the cavitation device in the disclosed manner, the three cavity portions 4350, 4352, 4354 may be formed about a plurality of adjacent axes such that they overlap to create a single tissue cavity 4348. As illustrated, a variety of cavity portion configurations may be combined to create a single desirable tissue cavity 4348.

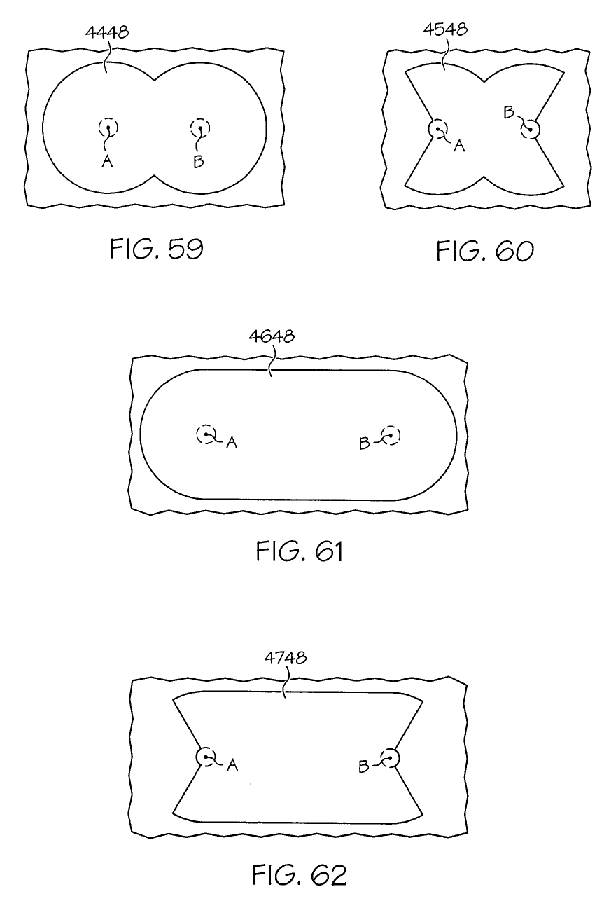

FIGS. 59-62 illustrate alternate versions of cross-sectional views taken along axis A-A of combined tissue cavities 4448, 4548, 4648, 4748 created by rotating a cavitation device about a first axis A-A and a second axis B-B. It will be appreciated that the axes A-A and B-B, and the corresponding cavities, are disclosed by way of example only, where the axes may be linear, non-linear, parallel, converging, or the like. As illustrated, any suitable number of cavities of any suitable shape may be connected to form a combined tissue cavity. Such combined tissue cavities may provide users with the ability to tailor a tissue cavity to their exact needs to provide high quality patient care.

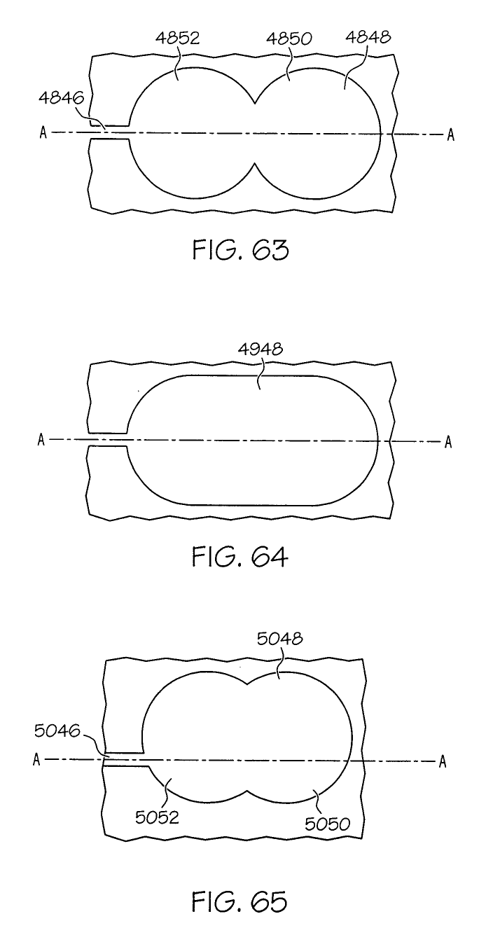

FIGS. 63-69 illustrate side views of versions of tissue cavities that may be created in accordance with cavitation devices, such as the cavitation device 900 of FIGS. 12A-C, disclosed herein. FIGS. 63-69 illustrate tissue cavities that combine multiple tissue cavity portions along a single axis, such as axis A-A, to form a combined tissue cavity. Creating combined cavities, such as those illustrated, may allow a user to tailor tissue cavities to maximize the therapeutic benefit. Tissue cavities created in the disclosed manner may be particularly well suited for receiving structural materials, such as PMMA, or for housing inflatable devices in accordance with kyphoplasty procedures.

FIG. 63 illustrates a side view of a combined tissue cavity 4848 having a first cavity section 4850 and a second cavity section 4852. The first cavity section 4850 and the second cavity section 4852 may be, for example, coaxial, substantially spherical, cavities formed along a single axis A-A. The combined tissue cavity 4848 may be formed with a single instrument such as, for example, with the cavitation device 900 disclosed in FIGS. 12A-12C. The combined tissue cavity 4848 may be formed, for example, by inserting the cavitation device in a closed position, or first shape, to the distal end of a pilot hole 4846. The cavitation device may then be laterally extended and rotated to form the first cavity section 4850. After creating the first cavity section 4850, the cavitation device may be retracted and drawn proximally along the pilot hole 4846 to a second position. Once situated, the cavitation device may again be laterally extended and rotated to create the second cavity section 4852. In this manner, a single cavitation device may be used to create multiple cavity portions of one or a plurality of geometries along a single axis. The multiple cavity portions may, for example, be connected via the pilot hole 4846 or with any other suitable channel, bore, or connection.

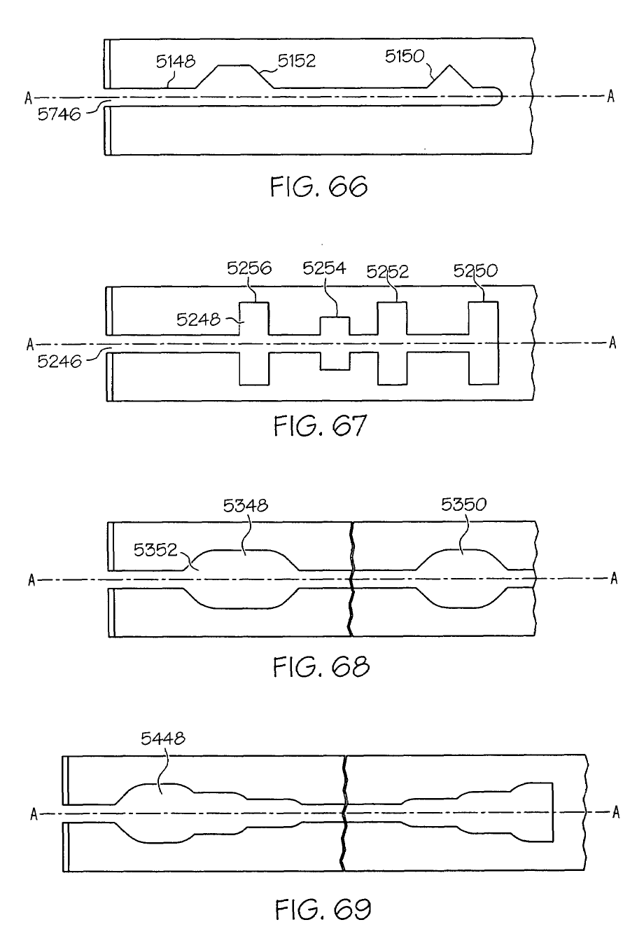

FIG. 64 illustrates one version of a combined tissue cavity 4948 combining multiple cavity portions to create a single cavity having a substantially uniform diameter. The combined tissue cavity 4948 may also be created, for example, by proximally or distally actuating a cavitation device as it is rotating to create a bore. FIG. 65 illustrates one version of a combined tissue cavity 5048 where the centroid of the first cavity portion 5050 and the second cavity portion 5052 are offset or otherwise not coaxial with the central axis A-A of the pilot hole 5046. FIG. 65 illustrates one example of a combined tissue cavity 5048 where cavities within tissue may be tailored such that particular regions are cut differently than others in accordance with cavitation devices disclosed herein.

FIG. 66 illustrates one version of a side view of a combined tissue cavity 5148 having a first lateral cavity portion 5150 and a second lateral cavity portion 5152. The combined tissue cavity 5148 may be created, for example with the cavitation device 900, disclosed in FIGS. 12A-12C, having the flexible cutting element 3820 disclosed in FIG. 41. For reference, the cross-sectional view of FIG. 66 along the A-A central axis may, for example, resemble the cross-section of FIG. 48. A pilot hole 5146 may be pre-drilled into tissue.

A first lateral cavity portion 5150 may be created in accordance with the first method disclosed with reference to FIG. 41, where the cavitation device may be inserted into the pilot hole 5146 adjacent the distal end of the pilot hole 5146. The flexible cutting element 3820 may then be actuated from an opened to a closed position repeatedly in a sawing motion to create the first lateral cavity portion 5150. In this version, the handle of the cavitation device may be held substantially static.

The cavitation device may then be closed, withdrawn proximally to a second position, and then operated in accordance with the second method disclosed with reference to FIG. 41. For example, the cavitation device may be opened such that the flexible cutting element is adjacent the bone surface. The cavitation device may then be translated axially, with the flexible cutting element is a generally static position, to create the second lateral cavity portion 5152. If a larger cavity is desired, the flexible cutting element may be extended further axially become again resuming the axial cutting motion of the cavitation device. As illustrated, multiple variations of cavity portions or sections may be combined into a single cavity.

FIG. 67 illustrates one version of a combined cavity 5248 having a plurality of substantially disk-shaped cavity portions 5250, 5252, 5254, 5256 connected with a pilot hole 5246. Cavity portions combined to form a combined tissue cavity, such as the cavity portions 5250, 5252, 5254, 5256 combined to form combined cavity 5248, may be of varying number, geometry, size, shape, and/or configuration. It will be appreciated that any suitable first cavity may be combined with any suitable second cavity to form a combined cavity.

FIG. 68 illustrates one version of a combined tissue cavity 5348 having a first cavity portion 5350 and a second cavity portion 5352. In the illustrated version, the first cavity portion 5350 is in one portion of a fractured bone and the second cavity portion 5352 is in a second portion of a fractured bone. By operating a cavitation device, such as the cavitation device 900 illustrated in FIGS. 12A-12C, in accordance with versions herein, the first cavity portion 5350 and the second cavity portion 5352 may be formed with a single cavitation device inserted through a single pilot hole 5346. The first cavity portion 5350 and the second cavity portion 5352 may be created for use in combination with an inflatable device. Such procedures for mending fractures may include, for example, those disclosed in copending U.S. Pat. Application 60/822,440 to Rossenwasser, et al., filed Aug. 15, 2006, which is herein incorporated by reference to the extent it is not limiting. The stepped tissue cavity 5448 illustrated in FIG. 69 may also be used, for example, in accordance with such procedures.

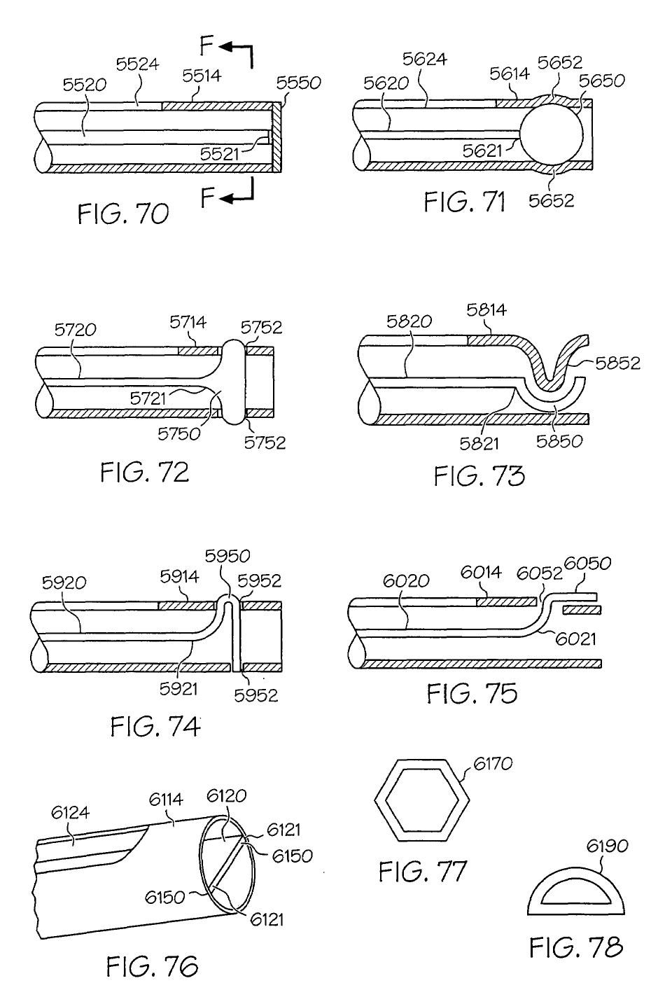

FIG. 70 illustrates a partial view of one version of the relationship between an insertion tube 5514 and a flexible cutting element 5520. In the illustrated version, the insertion tube 5514 includes a closure member 5550, such as a cap, to which a distal end 5521 of the flexible cutting element 5520 is fixed. The flexible cutting element 5520 may be opened through an aperture 5524 by compressing the flexible cutting element 5520 distally against the closure member 5550 such that the force causes at least a portion of the flexible cutting element 5520 to extend laterally. It will be appreciated that the cap, cap member, or closure member disclosed herein may be any suitable stop, movable member, closure device, closure assembly, distal cap, lateral cap, or the like.

FIG. 71 illustrates a partial view of one version of the relationship between an insertion tube 5614 and a flexible cutting element 5620. In the illustrated version, a spherical member 5650 is fixedly coupled to the distal end 5621 of the flexible cutting element 5620. The spherical member 5650 may be releasably coupled to the insertion tube 5614 by engaging the spherical member 5650 with catches 5652. The spherical member 5650 and/or the catches 5652 may be sufficiently flexible to enable coupling and decoupling. It will be appreciated that insertion tube variations discussed herein may be manufactured as fixed components.

In one version, the insertion tube 5614 is inserted into a pilot hole without the flexible cutting element 5620. Once the insertion tube 5614 is positioned, the flexible cutting element 5620 may be inserted until the spherical coupling member 5650 engages the catches 5652. The flexible cutting element 5620 may then be extended laterally through an aperture 5624 by compressing the flexible cutting element 5620 distally against the spherical coupling member 5650 such that the compression causes at least a portion of the flexible cutting element 5620 to extend laterally. The coupling between the spherical coupling member 5650 and the catches 5652 may be such that the threshold for laterally extending the flexible cutting element 5620 with compressive force may be less than that required to disengage the coupling. In one version, the flexible cutting element 5620 may be removed from the insertion tube 5614 while the insertion tube 5614 remains within the pilot hole by applying sufficient proximal force to disengage the coupling. In such a manner, multiple flexible cutting elements, such as the flexible cutting element 5620, may be inserted without having to remove the insertion tube 5614 from the pilot hole. The flexibility of such a device may facilitate precise cavity formation as a wide variety of blade types may be inserted without having to completely extract the instrument after the use of each flexible cutting element.

FIG. 72 illustrates a partial view of one version of the relationship between an insertion tube 5714 and a flexible cutting element 5720. In the illustrated version, a substantially disk-shaped coupling member 5750 is fixedly coupled to the distal end 5721 of the flexible cutting element 5720. The disk-shaped coupling member 5750 may be releasably coupled to the insertion tube 5714 by engaging the disk-shaped coupling member 5750 with catches 5752. The disk-shaped member 5750 may be sufficiently flexible to enable coupling and decoupling. It will be appreciated that, depending on the configuration of the insertion tube 5714, the disk-shaped coupling member 5750 may be any suitable shape, such as a polygonal shape.

FIG. 73 illustrates a partial view of one version of the relationship between an insertion tube 5814 and a flexible cutting element 5820. In the illustrated version, a clasp coupling member 5850 is fixedly coupled to the distal end 5821 of the flexible cutting element 5820. The clasp coupling member 5850 may be releasably coupled to the insertion tube 5814 by engaging the clasp coupling member 5850 with a detent 5852. The cup-shaped member 5850 and/or the detent 5852 may be sufficiently flexible to enable coupling and decoupling.

FIG. 74 illustrates a partial view of one version of the relationship between an insertion tube 5914 and a flexible cutting element 5920. In the illustrated version, a t-shaped coupling member 5950 is fixedly coupled to the distal end 5921 of the flexible cutting element 5920. The t-shaped coupling member 5950 may be releasably coupled to the insertion tube 5914 by engaging the t-shaped coupling member 5950 with catches 5952. The t-shaped coupling member 5950 may be sufficiently flexible to enable coupling and decoupling.

FIG. 75 illustrates a partial view of one version of the relationship between an insertion tube 6014 and a flexible cutting element 6020. In the illustrated version, a tongue or s-shaped coupling member 6050 is fixedly coupled to the distal end 6021 of the flexible cutting element 6020. The tongue or s-shaped coupling member 6050 may be releasably coupled to the insertion tube 6014 by engaging the tongue or s-shaped coupling member 6050 with a groove 6052. The tongue or s-shaped coupling member 6050 may be sufficiently flexible to enable coupling and decoupling from the groove 6052. It will be appreciated that the relationship between the insertion tubes and the flexible cutting elements disclosed herein is by way of example only and is not intended to be limiting.

FIG. 76 illustrates a partial view of one version of the relationship between an insertion tube 6114 and a flexible cutting element 6120. In the illustrated version, the distal end 6121 of the flexible cutting element 6120 is permanently coupled to the insertion tube 6114 with, for example, weld points 6150. The illustrated version of the insertion tube 6114 may allow the flexible cutting element 6120 to be laterally extended and retracted through the aperture 6124 while simultaneously allowing for the passage of matter, such as irrigation fluid, through the open distal end of the insertion tube 6114.

Referring to FIGS. 77 and 78 disclosed are alternate configurations of insertion tubes 6170 and 6190, respectively. The insertion tubes 6170, 6190 may have correspondingly configured shafts, flexible cutting elements, or any other suitable component. The insertion tubes 6170 and 6190 are disclosed by way of example to illustrate that any suitable configuration of elements of cavitation devices is contemplated.

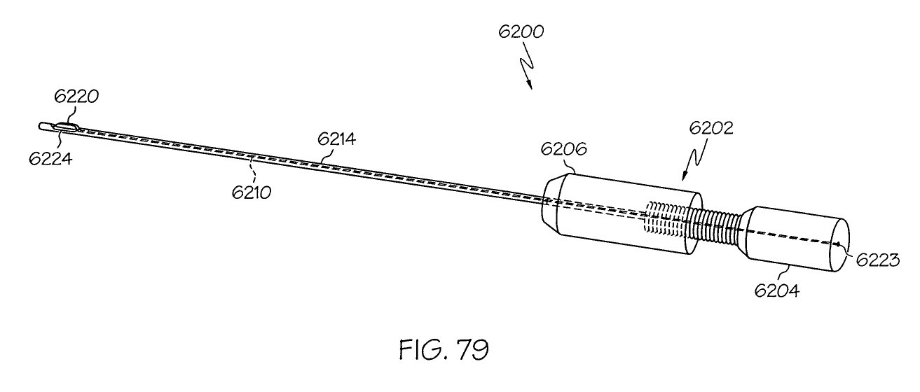

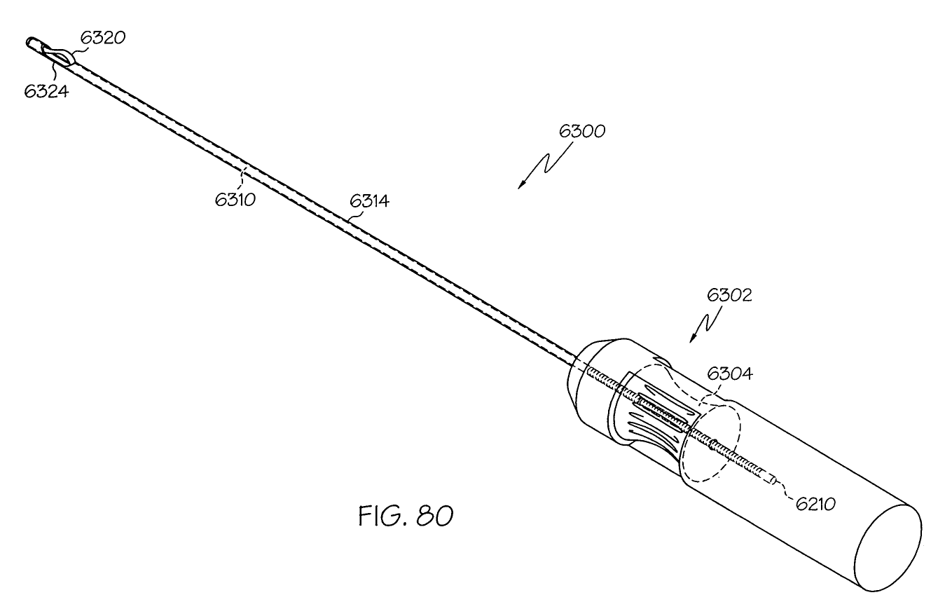

Referring to FIG. 79, disclosed is one version of a cavitation device 6200 that may be configured to laterally extend and retract a flexible cutting element 6220 from an aperture 6224 in an insertion tube 6214. In the illustrated version, the flexible cutting element 6220 is fixed to a shaft 6210 that extends proximally along the length of the cavitation device 6200 and is fixed, in the axial direction, at its proximal end 6223 to a knob 6204 of a handle 6202. The proximal end 6223 may be coupled with the knob 6204 such that it is freely rotatable relative to the knob 6204 such that axial motion of the knob 6204 will translate the shaft 6210 but rotational motion alone will not. The knob 6204 may be threadedly engaged with a base member 6206 such that manual rotation of the knob in one direction urges the shaft 6210 proximally and rotation of the knob 6204 in the other direction urges the shaft 6210 distally. Actuation of the shaft 6210, in the illustrated version, causes the flexible cutting element 6220 to laterally extend and retract through the aperture 6224. It will be appreciated, in an alternate embodiment, that the flexible cutting element may be coupled to the shaft such that it is rotatable relative thereto, where the proximal end of the shaft may be fixed both rotationally and axially to the knob.

The cavitation device 6200 may be operated, for example, by inserting the insertion tube 6214 into a pre-drilled pilot hole with the flexible cutting element 6220 substantially housed within the insertion tube 6214. Once positioned, the knob 6204 may be screwed into the base member 6206 whereby the shaft 6210 is urged distally. As the shaft 6210 is urged distally, the flexible cutting element 6220 may be urged laterally as it compresses against the distal end of the insertion tube 6214. After at least partially laterally extending the flexible cutting element 6220, the cavitation device 6200, or portions thereof, may be rotated to form or modify a tissue cavity. After completion of the tissue cavity, the knob may be rotated in the opposite direction such that the shaft 6210 attached thereto is drawn proximally. Drawing the shaft 6210 proximally will, in the illustrated version, retract the flexible cutting element 6220 into the aperture 6224 for removal from the pilot hole. It will be appreciated that all versions of the cavitation device disclosed herein may be operated in the disclosed manner or in any other suitable manner.

In an alternate version, a cavity may be formed with the cavitation device 6200 by inserting the insertion tube 6214 into a pre-drilled pilot hole with the flexible cutting element 6220 substantially housed within the insertion tube 6214. Once positioned, the knob 6204 may be screwed into the base member 6206 whereby the shaft 6210 is urged distally. As the shaft 6210 is urged distally, the flexible cutting element 6220 may be urged laterally as it compresses against the distal end of the insertion tube 6214. After laterally extending the flexible cutting element 6220 until contact is made with the tissue the cavitation device 6200 may be translated axially in a sawing motion to create a cavity. To create a larger tissue cavity, the knob may be rotated in the same direction such that the flexible cutting element is again extended laterally adjacent the bone tissue. The cavitation device 6200 may then, as before, be translated axially. It will be appreciated that versions of the cavitation device disclosed herein may be used in accordance with any suitable method of cavity formation.

In an alternate version, a cavity may be formed with the cavitation device 6200 by inserting the insertion tube 6214 into a pre-drilled pilot hole with the flexible cutting element 6220 substantially housed within the insertion tube 6214. Once positioned, the knob 6204 may be screwed into the base member 6206 whereby the shaft 6210 is urged distally. As the shaft 6210 is urged distally, the flexible cutting element 6220 may be urged laterally as it compresses against the distal end of the insertion tube 6214. After laterally extending the flexible cutting element 6220 until contact is made with the tissue, the cavitation device 6200, or cutting portions thereof, may be rotated to form a cavity. To create a larger tissue cavity, the knob may be rotated in the same direction such that the flexible cutting element is again extended laterally adjacent the bone tissue. The cavitation device 6200 may then, as before, be rotated. It will be appreciated that versions of the cavitation device disclosed herein may be used in accordance with any suitable method of cavity formation.

Referring to FIG. 80, disclosed is an alternate version of a cavitation device 6300 that may be configured to laterally extend and retract a flexible cutting element 6320 from an aperture 6324 in an insertion tube 6314. In the illustrated version, the flexible cutting element 6320 is fixed to a threaded shaft 6310 that extends proximally along the length of the cavitation device 6300. In the illustrated version, the threaded shaft 6310 engages a threaded rotational actuation member 6304 that is rotatable within a handle 6302. The relationship between the threaded shaft 6310 and the rotational actuation member 6304 may be such that manual rotation of the rotational actuation member 6304 in one direction urges the threaded shaft 6310 proximally and rotation of the rotational actuation member 6304 in the other direction urges the threaded shaft 6310 distally. Actuation of the shaft 6310, in the illustrated version, causes the flexible cutting element 6320 to laterally extend and retract through the aperture 6324 and is self-latching.

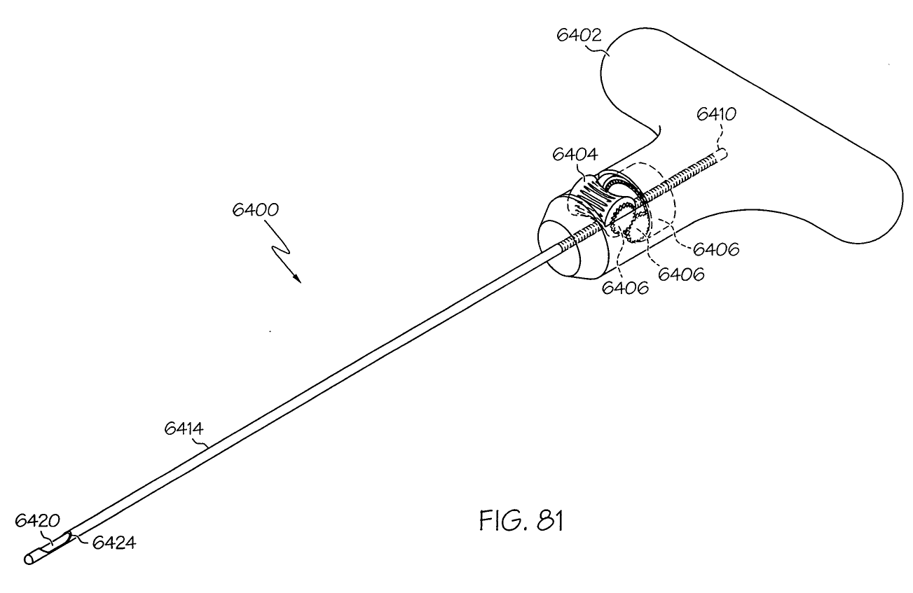

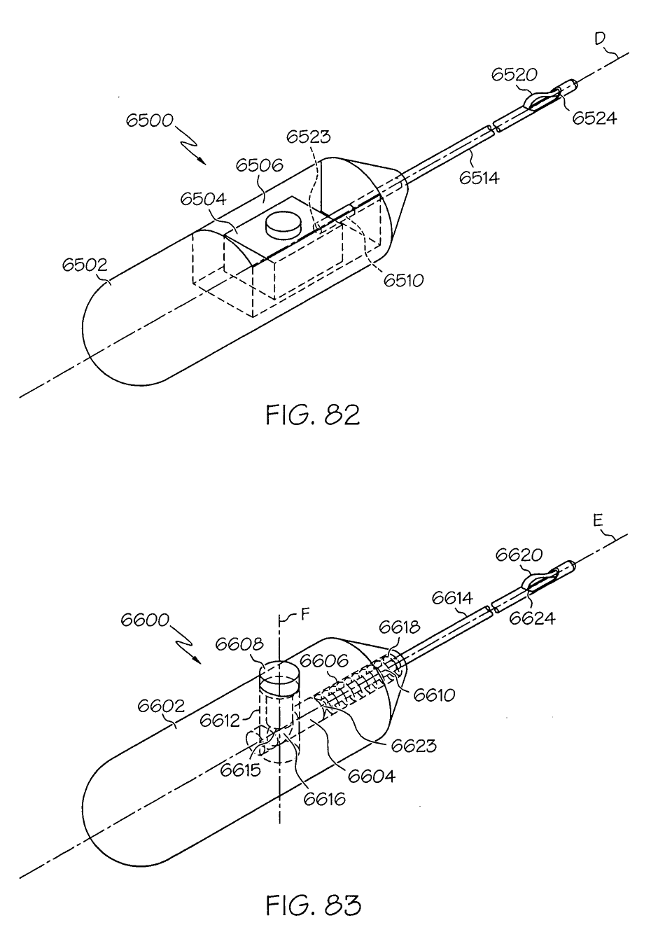

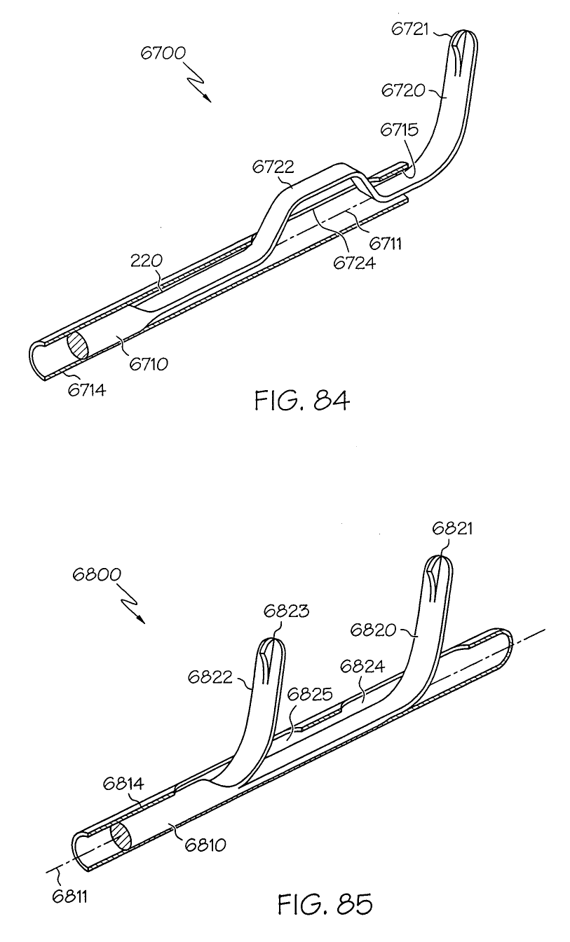

Referring to FIG. 81, disclosed is an alternate version of a cavitation device 6400 that may be configured to laterally extend and retract a flexible cutting element 6420 from an aperture 6424 in an insertion tube 6414. In the illustrated version, the flexible cutting element 6420 is coupled with a threaded shaft 6410 that extends proximally along the length of the cavitation device 6400. In the illustrated version, the threaded shaft 6410 is associated with a rotational actuation member 6404 via a gear assembly 6406 housed within a handle 6402. The relationship between the threaded shaft 6410 and the rotational actuation member 6404 may be such that manual rotation of the rotational actuation member 6404 in one direction urges the threaded shaft 6410 proximally and rotation of the rotational actuation member 6404 in the other direction urges the threaded shaft 6410 distally. Actuation of the shaft 6410, in the illustrated version, causes the flexible cutting element 6420 to laterally extend and retract through the aperture 6424 and is self-latching.

Referring to FIG. 82, disclosed is an alternate version of a cavitation device 6500 that may be configured to laterally extend and retract a flexible cutting element 6520 from an aperture 6524 in an insertion tube 6514. In the illustrated version, the flexible cutting element 6520 is coupled with a shaft 6510 that extends proximally along the length of the cavitation device 6500 and is fixed at its proximal end 6523 to a slide 6504 operably configured to translate within a track 6506 in a handle 6502. The slide 6504 may be translated within the track 6506 along the axes D-D such that manual actuation of the slide 6504 in the distal direction urges the shaft 6510 distally, thereby laterally extending the flexible cutting element 6520, and actuation of the slide 6504 in the proximal direction urges the shaft 6510 proximally, thereby retracting the flexible cutting element 6520. The cavitation device 6500 an element, such as a ratchet (not shown), with which the slide and/or flexible cutting element may be moved in degrees.

Referring to FIG. 83, disclosed is an alternate version of a cavitation device 6600 that may be configured to laterally extend and retract a flexible cutting element 6620 from an aperture 6624 in an insertion tube 6614. In the illustrated version, the flexible cutting element 6620 is coupled with a shaft 6610 that extends proximally along the length of the cavitation device 6600 and is fixed at its proximal end 6623 to a first cylinder 6604 operably configured to translate along axis E-E within a longitudinal track 6606 in a handle 6602, where the first cylinder 6604 is biased proximally by a spring 6618 retained within the longitudinal track 6606. The handle 6602 further includes a second cylinder 6608 operably configured to translate along axes F-F within an axial track 6612. The second cylinder 6608 includes an abutment surface 6615 operably configured to engage an angled abutment surface 6616 of the first cylinder 6604. The first cylinder 6604 may be translated distally within the track 6606 when the second cylinder 6608 is manually depressed, where depressing the second cylinder 6608 engages the abutment surface 6615 and the angled abutment surface 6616 thereby urging the first cylinder 6604 distally. It will be appreciated that the illustrated surfaces may be provided with any suitable configuration. The first cylinder 6604 may be returned proximally to a resting position by the spring 6618 when the second cylinder 6608 is released. Actuation of the first cylinder 6604 in the distal direction urges the shaft 6610 distally, thereby laterally extending the flexible cutting element 6620, and actuation of the first cylinder 6604 in the proximal direction urges the shaft 6610 proximally, thereby retracting the flexible cutting element 6620. The cavitation device 6600 may also be provided with a latching mechanism (not shown) to secure the flexible cutting element and/or to indicate the shape or position of the flexible cutting element to the user.

FIG. 84 shows an alternate version of a cavitation device 6700, comprising a shaft 6710, a first flexible cutting element 6720 having a free end 6721, and a second flexible cutting element 6722. The flexible cutting elements 6720, 6722 may be formed from, for example, stainless steel. In the illustrated version, the shaft 6710 has a longitudinal axis 6711. When the second flexible cutting element 6722 is aligned with the lateral aperture 6724 of the insertion tube 6714 the second flexible cutting element 6722 may project outward or laterally from the longitudinal axis. The second flexible cutting element 6722 may be retained in a retracted, or first shape, while in the insertion tube 6714 prior to opening into a laterally projecting configuration, or second shape, as illustrated. The second flexible cutting element may open outwardly into a remember shape upon introduction to the aperture 6724, may be projected when exposed to heat, may be uncoiled, or may otherwise be expanded laterally. The second flexible cutting element 6722 may have a bias toward a “remembered” second shape, in which the flexible cutting element 6722 extends or projects away from the longitudinal axis 6711 of the shaft 6710 in the general shape of a curvilinear arch, as illustrated. Once in the second shape, rotation of the second flexible cutting element 6722 in a clockwise and/or counterclockwise direction may be used to form or modify a tissue cavity.

Still referring to FIG. 84, as the first flexible cutting element 6720 extends past the distal end 6715 of the insertion tube 6714, the first flexible cutting element 6720 may be laterally or outwardly projected. Upon projection, the first flexible cutting element 6720 may be converted into a projected, or second shape, in which both the first flexible cutting element 6720 and the second flexible cutting element 6722 are laterally projected. When in the second shape, as illustrated, two tissue cavity portions may be created simultaneously for placement of, for example, a vertebroplasty or kyphoplasty balloon. It will be appreciated that any mode of transition from a first shape for the flexible cutting elements is contemplated. It is further contemplated that there be a plurality of flexible cutting elements positioned at about any suitable location of the cavitation device 6700. It is further contemplated that the first flexible cutting element and the second flexible cutting element may respond to varying projection stimuli where, for example, the first flexible cutting element may project outwardly when introduced to a first temperature and the second flexible cutting element may project outwardly when introduced to a second temperature. In this manner, for example, the first flexible cutting element and the second flexible cutting element may open independently from one another.

FIG. 85 shows an alternate version of a cavitation device 6800, comprising a shaft 6810, a first flexible cutting element 6820 having a free end 6821, and a second flexible cutting element 6822 having a free end 6823. The flexible cutting elements 6820, 6822 may be formed from, for example, stainless steel. In the illustrated version, the shaft 6810 has a longitudinal axis 6811. When the first flexible cutting element 6820 is aligned with the first lateral aperture 6824 of the insertion tube 6814, the first flexible cutting element 6820 may project outward or laterally from the longitudinal axis 6811. The first flexible cutting element 6820 may be retained in a retracted, or first shape, while housed within the insertion tube 6814 prior to opening into a laterally projecting configuration, or second shape, as illustrated. The first flexible cutting element 6820 may open outwardly into a remembered shape upon introduction to the first lateral aperture 6824, may be projected when exposed to heat, may be uncoiled, or may otherwise be expanded laterally. The first flexible cutting element 6820 may have a bias toward a “remembered” second shape, in which the first flexible cutting element 6820 extends or projects away from the longitudinal axis 6811 of the shaft 6810 in the general shape of a curvilinear projection, as illustrated. Once in the second shape, rotation of the first flexible cutting element 6820 in a clockwise and/or counterclockwise direction may be used to form or modify a tissue cavity.

When the second flexible cutting element 6822 is aligned with the second lateral aperture 6825 of the insertion tube 6814, the second flexible cutting element 6822 may project outward or laterally from the longitudinal axis 6811. The second flexible cutting element 6822 may be retained in a retracted, or first shape, while housed within the insertion tube 6814 prior to opening into a laterally projecting configuration, or second shape, as illustrated. The second flexible cutting element 6822 may open outwardly into a remember shape upon introduction to the second lateral aperture 6825, may be projected when exposed to heat, may be uncoiled, or may otherwise be expanded laterally. The second flexible cutting element 6822 may have a bias toward a “remembered” second shape, in which the flexible cutting element 6822 extends or projects away from the longitudinal axis 6811 of the shaft 6810 in the general shape of a curvilinear arch, as illustrated. Once in the second shape, rotation of the second flexible cutting element 6822 in a clockwise and/or counterclockwise direction may be used to form or modify a tissue cavity.

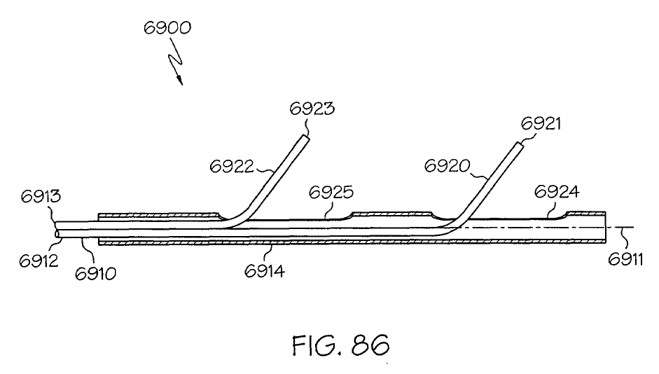

FIG. 86 shows an alternate version of a cavitation device 6900, comprising a shaft 6910 having a first shaft portion 6912 and a second shaft portion 6913. In the illustrated version, the first shaft portion 6912 is coupled with a first flexible cutting element 6920 having a free end 6921, and the second shaft portion 6913 is coupled with a second flexible cutting element 6922 having a free end 6923. The shaft portions 6912, 6913 may be adjacent hemispheres configured such that the shaft 6910 is substantially cylindrical. The shaft portions 6912, 6913 may be movable relative to one another such that the flexible cutting elements 6920, 6922 may be actuated independently. The flexible cutting elements 6920, 6922 may be formed from, for example, stainless steel. The shaft 6910 has a longitudinal axis 6911.

Still referring to FIG. 86, when the first flexible cutting element 6920 is aligned with the first lateral aperture 6924 of the insertion tube 6914, the first flexible cutting element may project outward or laterally from the longitudinal axis 6911. The first flexible cutting element 6920 may be retained in a retracted, or first shape, while housed within the insertion tube 6914 prior to opening into a laterally projecting configuration, or second shape, as illustrated. The first flexible cutting element 6920 may be introduced to the first lateral aperture 6924 with the first shaft portion 6912 and may be opened outwardly into a remembered shape, may be projected laterally when exposed to heat, or may otherwise be projected laterally. The first flexible cutting element 6920 may have a bias toward a “remembered” second shape in the general shape of a curvilinear projection, as illustrated. Once in the second shape, rotation of the first flexible cutting element 6920 in a clockwise and/or counterclockwise direction may be used to form or modify a tissue cavity.

When the second flexible cutting element 6922 is aligned with the second lateral aperture 6925 of the insertion tube 6914, the second flexible cutting element 6922 may project outward or laterally from the longitudinal axis 6911. The second flexible cutting element 6922 may be retained in a retracted, or first shape, while in the insertion tube 6914 prior to opening into a laterally projecting configuration, or second shape, as illustrated. The second flexible cutting element 6922 may be introduced to the second lateral aperture 6925 with the second shaft portion 6913 and may be opened outwardly into a remembered shape, may be projected laterally when exposed to heat, or may otherwise be projected laterally. The second flexible cutting element 6922 may have a bias toward a “remembered” second shape in the general shape of a curvilinear projection, as illustrated. Once in the second shape, rotation of the second flexible cutting element 6922 in a clockwise and/or counterclockwise direction may be used to form or modify a tissue cavity.

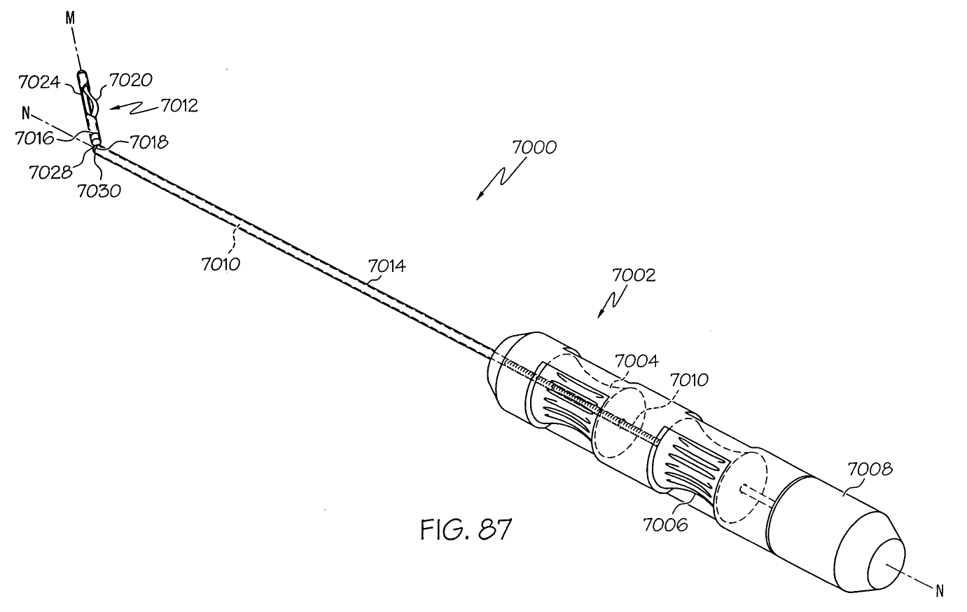

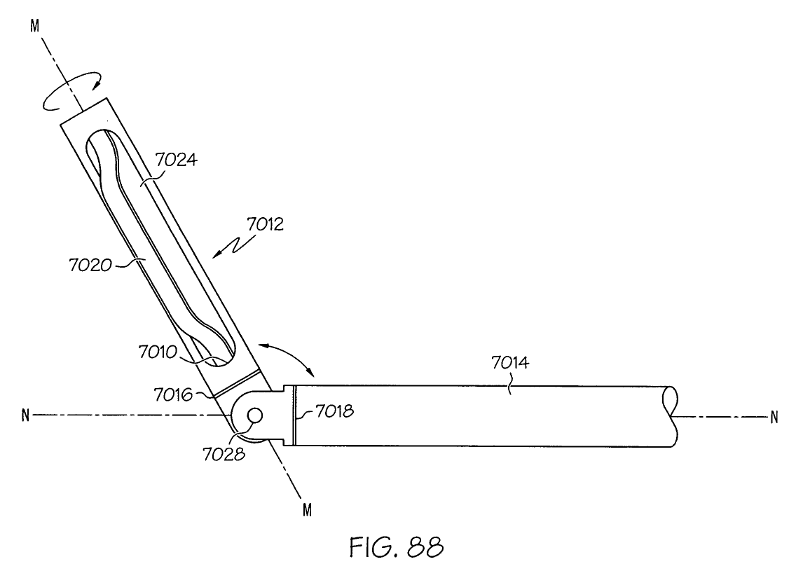

Referring to FIGS. 87-88, disclosed is an alternate version of a cavitation device 7000 that may be configured to laterally extend and retract a flexible cutting element 7020 from an aperture 7024 in an insertion tube 7014. In the illustrated version, the flexible cutting element 7020 is fixed to a threaded shaft 7010 that extends proximally along the length of the cavitation device 7000. In the illustrated version, the threaded shaft 7010 engages a threaded rotational actuation member 7004 that is rotatable within an actuator or handle 7002. The relationship between the threaded shaft 7010 and the rotational actuation member 7004 may be such that manual rotation of the rotational actuation member 7004 in one direction urges the threaded shaft 7010 proximally and rotation of the rotational actuation member 7004 in the other direction urges the threaded shaft 7010 distally. Actuation of the shaft 7010, in the illustrated version, causes the flexible cutting element 7020 to laterally extend and retract through the aperture 7024 and is self-latching.

Still referring to FIGS. 87-88, the cavitation device 7000 includes an end effector 7012 configured for articulation. The end effector 7012 may be configured for articulation rotationally, laterally, pivotally, or in any other suitable, direction, mode, or manner. For example, the end effector 7012 may be pivotable about an angular joint 7028, where the pivotal motion is controlled by, for example, a rotational actuation member 7006 coupled with the angular joint 7028, such that rotational motion of the rotational actuation member 7006 translates into pivotal motion at the end effector 7012. The end effector 7012 further includes a rotational joint 7016 configured to rotate the end effector 7012 about the M-M axis, or central axis thereof. Rotational motion about the rotational joint 7016 may be provided via a rotational actuation member 7008, where rotation of the rotational actuation member 7008 may correspondingly translate into rotational motion of the end effector 7012. The insertion tube 7014 may include a rotational joint 7018 that may be coupled with an actuator (not shown) such that an additional degree of freedom is provided. It will be appreciated that any suitable articulation, rotation, or movement of the cavitation device is contemplated, where any suitable number or configuration of articulations may be provided.

It will be appreciated that any suitable number of flexible cutting elements having any suitable configuration may be provided at any suitable location about the cavitation device. For example, a plurality of flexible cutting elements may be disposed at intervals, axially, and also disposed radially about the longitudinal axis. Any combination of distally positioned and axially positioned flexible cutting elements is contemplated. It will be further appreciated that any suitable mode of opening or transitioning to a second shape is contemplated.

The versions presented in this disclosure are examples. Those skilled in the art can develop modifications and variants that do not depart from the spirit and scope of the disclosed cavitation devices and methods. For example, there are instances where an insertion tube is not required and a pilot hole in bone tissue is appropriate for passage to the cavitation site. Disclosed flexing methods or devices for biasing the flexible cutting elements to move from a first shape to a second shape include elastic deformation, thermal shape-memory, centrifugal force, and force applied through a tension cable. Although these are considered in the examples separately, cavitation devices of the present invention may include additional methods of movement and a combination of two or more of these methods. Those skilled in the art will understand that markings on the shaft of a cavitation device of the invention may be used for indicating depth of insertion and that an additional fitting on the shaft may be used to limit the depth of insertion. Thus, the scope of the invention should be determined by the appended claims and their legal equivalents, rather than by the examples given.

What is claimed is:

1. A medical device for cavity formation comprising:
(a) a shaft member, extending along a longitudinal axis, associated with a flexible cutting element comprising;
(i) a first shape at a first time for placement within an anatomical structure, and
(ii) a second shape at a second time, wherein the second shape of the flexible cutting element is operably configured to be outwardly deformed to form a cavity,
(b) an insertion tube, wherein the insertion tube is operably configured to retain at least a portion of the shaft member;
(c) a lateral aperture, wherein the lateral aperture is provided in the insertion tube and is operably configured to accept passage of the second shape of the flexible cutting element therethrough; and
(d) an actuator, the actuator being coupled with the insertion tube, wherein the actuator is operably configured to deform the flexible cutting element from the first shape to the second shape.

2. The medical device of claim 1, wherein the second shape is a remembered shape.

3. The medical device of claim 1, wherein the second shape is achieved with centrifugal force.

4. The medical device of claim 1, wherein the second shape is achieved with a temperature change.

5. The medical device of claim 1, wherein the flexible cutting element is a leaf spring.

6. The medical device of claim 1, wherein the flexible cutting element comprises a cutting tip.

7. The medical device of claim 1, wherein the shaft member includes a fluid channel.

8. The medical device of claim 1, wherein the flexible cutting element is offset from the longitudinal axis in the first shape.

9. The medical device of claim 1, wherein the cavity formation is performed manually.

10. The medical device of claim 1, wherein the cavity formation is directed to the spine.

11. The medical device of claim 1, wherein the medical device is configured for use in a procedure selected from the group consisting of the treatment of bone fracture, the prevention of bone fracture, joint fusion, implant fixation, tissue harvesting, bone tissue harvesting, removal of diseased soft tissue, removal of diseased hard tissue, general soft tissue removal, general hard tissue removal, vertebroplasty, kyphoplasty, and combinations thereof.

12. The medical device of claim 1, wherein the insertion tube is configured with a cross-section selected from the group consisting of, a polygon, circular, a hexagon, a pentagon, a hemisphere, a curvilinear cross-section, and combinations thereof.

13. A method of tissue cavity formation comprising:
providing a medical device comprising;
(a) a shaft member, extending along a longitudinal axis, associated with a flexible cutting element comprising;
(i) a first shape at a first time for placement within an anatomical structure, and
(ii) a second shape at a second time, wherein the second shape of the flexible cutting element is outwardly deformed to form a cavity,
(b) an insertion tube, wherein the insertion tube is operably configured to retain at least a portion of the shaft member,
(c) a lateral aperture, wherein the lateral aperture is provided in the insertion tube and is operably configured to accept passage of the second shape of the flexible cutting element therethrough, and
(d) an actuator, the actuator being coupled with the insertion tube, wherein the actuator is operably configured to deform the flexible cutting element from the first shape to the second shape,
inserting the flexible cutting element in the first shape into tissue;
deforming the flexible cutting element into the second shape; and
forming a tissue cavity.

14.-74. (canceled)

75. A medical device for cavity formation comprising:
(a) a shaft member, extending along a longitudinal axis, associated with a flexible cutting element comprising;
(i) a deformable elongated body having a first end and a second end, wherein the first end is coupled with the shaft member,
(ii) a first shape at a first time configured for placement within an anatomical structure, and
(iii) a second shape at a second time, wherein the second shape of the flexible cutting element is outwardly deformed to form a cavity, wherein the second shape comprises a first concavity and a first convexity and a second concavity and a second convexity,
(b) an insertion tube, the insertion tube being configured to retain at least a portion of the shaft member, wherein the second end of the flexible cutting element is coupled with the insertion tube; and
(c) a lateral aperture, the lateral aperture having a transverse axis, the lateral aperture being operably configured to accept passage of the second shape of the flexible cutting element therethrough; and
(d) an actuator, the actuator being coupled with the insertion tube, wherein the actuator is operably configured to deform the flexible cutting element from the first shape to the second shape.

76. The medical device of claim 75, wherein the first concavity and the first convexity are provided by the arch formed when the flexible cutting element is laterally outwardly deformed into the second shape.

77. The medical device of claim 76, wherein the second concavity and the second convexity are configured with reference to the longitudinal axis.

78. The medical device of claim 77, wherein the second convexity may be used to cut tissue in a first direction and the second concavity may be used to cut tissue in a second direction.

79. The medical device of claim 78, wherein the first direction is counterclockwise and the second direction is clockwise.

80.-131. (canceled)

132. A medical device for cavity formation comprising:
(a) a shaft member extending along a longitudinal axis and including a flexible cutting element having;
(i) a deformable elongated body, having a first end and a second end, comprising,
(A) a first edge,
(B) a second edge,
(C) a top surface, the top surface having a top surface effect operably configured to form a lateral cavity, and
(D) a bottom surface,
(ii) a first shape for placement within an anatomical structure, and
(iii) a second shape, wherein the second shape of the flexible cutting element is outwardly deformed to form a cavity,
(b) an insertion tube, the insertion tube configured to retain at least a portion of the shaft member, wherein the second end of the flexible cutting element is coupled with the insertion tube;
(c) an aperture, wherein the aperture is provided in the insertion tube and is operably configured to accept passage of the second shape of the flexible cutting element therethrough; and
(d) an actuator, the actuator being coupled with the insertion tube, wherein the actuator is operably configured to deform the flexible cutting element from the first shape to the second shape.

133. The medical device of claim 132, wherein the top surface is operably configured to cut tissue.

134. The medical device of claim 133, wherein the top surface effect includes at least one projection.

135. The medical device of claim 133, wherein the top surface effect includes texture.

136. The medical device of claim 132, wherein the first edge and the second edge are configured to form a cavity with rotational motion and the top surface is configured to cut tissue with axial motion.

137. The medical device of claim 132, wherein the first edge and the second edge are configured to form a cavity with rotational motion and the top surface is configured to cut tissue with lateral motion.

138. The medical device of claim 132, wherein the shaft member includes a fluid channel.

139. The medical device of claim 132, wherein the medical device is configured for use in a procedure selected from the group consisting of the treatment of bone fracture, the prevention of bone fracture, joint fusion, implant fixation, tissue harvesting, bone tissue harvesting, removal of diseased soft tissue, removal of diseased hard tissue, general soft tissue removal, general hard tissue removal, vertebroplasty, kyphoplasty, and combinations thereof.

140. The medical device of claim 132, wherein the insertion tube is configured with a cross-section selected from the group consisting of a polygon, circular, a hexagon, a pentagon, a hemisphere, a curvilinear cross-section, and combinations thereof.

141. A method for lateral cavity formation comprising the steps of:
providing a medical device for cavity formation comprising:
(a) a shaft member, extending along a longitudinal axis, associated with a flexible cutting element comprising;
(i) a deformable elongated body, having a first end and a second end, comprising,
(A) a first edge,
(B) a second edge,
(C) a top surface, the top surface having a top surface effect operably configured to form a lateral cavity, and
(D) a bottom surface, and
(ii) a first shape for placement within an anatomical structure, and
(iii) a second shape, wherein the second shape of the flexible cutting element is outwardly deformed to form a cavity,
(b) an insertion tube, the insertion tube configured to retain at least a portion of the shaft member, wherein the second end of the flexible cutting element is coupled with the insertion tube;
(c) an aperture, wherein the aperture is provided in the insertion tube and is operably configured to accept passage of the second shape of the flexible cutting element therethrough; and
(d) an actuator, the actuator being coupled with the insertion tube, wherein the actuator is operably configured to deform the flexible cutting element from the first shape to the second shape; and
cutting with the top surface such that a tissue cavity is formed.

142. The method of claim 141, wherein the cutting step comprises maintaining the insertion tube in a substantially stationary position while deforming the flexible cutting element from a first shape to a second shape repeatedly to laterally cut tissue.

143. The method of claim 141, wherein the cutting step comprises;
extending the flexible cutting element into the second shape at a first lateral distance;
securing the flexible cutting element in the second shape; and
translating the instrument in a generally longitudinal direction to form a first cavity portion.

144. The method of claim 143, further comprising the steps of;
laterally extending the flexible cutting element to a second lateral distance, wherein the second lateral distance is greater than the first lateral distance; and
translating the instrument in a generally longitudinal direction to form a second cavity portion.

145. The method of claim 141, wherein the flexible cutting element is configured for rotational and lateral tissue cutting.

146. A method of forming a tissue cavity comprising:
providing a medical device for cavity formation comprising;
(a) a shaft member, extending along a longitudinal axis, associated with a flexible cutting element comprising;
(i) a first shape at a first time for placement within an anatomical structure, and
(ii) a second shape at a second time, wherein the second shape of the flexible cutting element is outwardly deformed to form a cavity,
(b) an insertion tube, wherein the insertion tube is operably configured to retain at least a portion of the shaft member;
(c) a lateral aperture, wherein the lateral aperture is provided in the insertion tube and is operably configured to accept passage of the second shape of the flexible cutting element therethrough;
(d) an actuator, the actuator being coupled with the insertion tube, wherein the actuator is operably configured to deform the flexible cutting element from the first shape to the second shape; and
cutting a first tissue cavity portion by rotating the flexible cutting element in the second shape about a central axis less than 360 degrees.

147. The method of claim 146, wherein the first tissue cavity portion is hemispherical.

148. The method of claim 146, further comprising the step of cutting a second tissue cavity portion.

149. The method of claim 148, wherein the first tissue cavity portion and the second tissue cavity portion are differently configured.

150. The method of claim 148, wherein the first tissue cavity portion and the second tissue cavity portion are created simultaneously.

151. The method of claim 148, wherein the first tissue cavity portion and the second tissue cavity portion are created at a first time and at a second time.

152. The method of claim 148, where the first tissue cavity portion is created with rotational cutting and the second tissue cavity portion is created with lateral cutting.

153. A method of cavity formation comprising the steps of:
providing a tissue cutting instrument comprising;
(a) a shaft member, extending along a longitudinal axis, associated with a flexible cutting element having;
(i) a first cutting element having a first surface effect,
(ii) a second cutting element having a second surface effect,
(iii) a first shape for placement within an anatomical structure, and
(iv) a second shape, wherein the second shape of the flexible cutting element is outwardly deformed to form a cavity,
(b) an insertion tube, wherein the insertion tube is operably configured to retain at least a portion of the shaft member;
(c) an aperture, wherein the aperture is provided in the insertion tube and is operably configured to accept passage of the second shape of the flexible cutting element therethrough; and
(d) an actuator, the actuator being coupled with the insertion tube, wherein the actuator is operably configured to deform the flexible cutting element from the first shape to the second shape;
forming a first tissue cavity portion about a first axis; and
forming a second tissue cavity portion about a second axis.

154. The method of claim 153, wherein the first axis and the second axis are not coaxial.

155. The method of claim 153, wherein the axes are substantially parallel.

156. The method of claim 153, wherein the axes are intersecting.

157. The method of claim 153, wherein the axes are aligned.

158. The method of claim 153, wherein the geometry of each of the cavity portions are selected from the group consisting of a hemispherical configuration, a round configuration, a cylindrical configuration, an ovular configuration, a polygonal configuration, an amorphous configuration, an axisymmetrical configuration, and combinations thereof.

159. The method of claim 153, further comprising the step of creating a combined tissue cavity by connecting the first tissue cavity portion with the second tissue cavity portion.

160. The method of claim 153, wherein the first tissue cavity portion and the second tissue cavity portion are substantially similar.

161. The method of claim 153, wherein the first tissue cavity portion and the second tissue cavity portion are differently configured.

162. The method of claim 153, wherein first tissue cavity portion and the second tissue cavity portion are formed simultaneously.

163. The method of claim 153, wherein the first tissue cavity portion and the second tissue cavity portion are formed at a first time and at a second time.

164. The method of claim 153, further comprising the step of forming a third tissue cavity portion about a third axis, wherein the third tissue cavity portion is connected with the first tissue cavity portion and the second tissue cavity portion such that a combined tissue cavity is formed.

165. The method of claim 164, wherein the axes are substantially parallel.

166. The method of claim 164, wherein the axes are intersecting.

167. The method of claim 164, wherein the axes are aligned.

168. The method of claim 164, wherein the geometry of each of the cavity portions are selected from the group consisting of a hemispherical configuration, a round configuration, a cylindrical configuration, an ovular configuration, a polygonal configuration, an amorphous configuration, an axisymmetrical configuration, and combinations thereof.

169. The method of claim 164, further comprising the step of forming a plurality of tissue cavity portions about a plurality of axes, wherein the plurality of tissue cavity portions are connected such that a combined tissue cavity is formed.

170. The method of claim 164, wherein the axes are skewed.

171. The method of claim 153, wherein the axes are skewed.

172. A method of cavity formation comprising the steps of:
providing a tissue cutting instrument comprising;
(a) a shaft member, extending along a longitudinal axis, associated with a flexible cutting element having;
(i) a first cutting element having a first surface effect,
(ii) a second cutting element having a second surface effect,
(iii) a first shape for placement within an anatomical structure, and
(iv) a second shape, wherein the second shape of the flexible cutting element is outwardly deformed to form a cavity,
(b) an insertion tube, wherein the insertion tube is operably configured to retain at least a portion of the shaft member;
(c) an aperture, wherein the aperture is provided in the insertion tube and is operably configured to accept passage of the second shape of the flexible cutting element therethrough; and
(d) an actuator, the actuator being coupled with the insertion tube, wherein the actuator is operably configured to deform the flexible cutting element from the first shape to the second shape;
forming a first tissue cavity portion about a first axis; and
forming a second tissue cavity portion about the first axis.

173. The method of claim 172, wherein the first tissue cavity portion is connected to the second cavity portion.

174. The method of claim 173, wherein the first tissue cavity portion is connected to the second cavity portion via a pilot hole.

175. The method of claim 172, wherein the first tissue cavity portion is at least partially distinct from the second cavity portion.

176. The method of claim 172, wherein the first tissue cavity portion and the second tissue cavity portion are combined to form a substantially uniform combined tissue cavity.

177. The method of claim 172, wherein the first axis is offset from the centroid of the tissue cavity portions.

178. The method of claim 172, wherein the first tissue cavity portion is configured differently from the second cavity portion.

179. The method of claim 172, wherein the first tissue cavity portion is a lateral tissue cavity.

180. The method of claim 172, wherein the first tissue cavity portion is a rotational tissue cavity.

181. The method of claim 180, wherein the second tissue cavity portion is a lateral tissue cavity.

182. The method of claim 172, wherein the first tissue cavity portion is in a first bone fracture portion and the second tissue cavity is in a second bone fracture portion, wherein the first and the second tissue cavity portions are configured to facilitate healing between the first and the second bone fracture portions.

183. The method of claim 182, wherein the first tissue cavity portion and the second tissue cavity portion are stepped.

184. The method of claim 172, wherein the first tissue cavity portion is an axial tissue cavity.

185. The method of claim 172, wherein the first tissue cavity portion and the second tissue cavity portion are a cavity portion selected from the group consisting of a lateral tissue cavity, an axial tissue cavity, a rotational tissue cavity, and combinations thereof.

186.-216. (canceled)
